(19) United States
(12) Patent Application Publication
(10) Pub. No.: US 2011/0091903 A1
(43) Pub. Date: Apr. 21, 2011

(54) METHOD OF ANALYZING A SAMPLE FOR A BACTERIUM USING DIACETYLENE-CONTAINING POLYMER SENSOR

(76) Inventors: G. Marco Bommarito, Stillwater, MN (US); Sridhar V. Dasaratha, Bangalore (IN); Brinda B. Lakshmi, Woodbury, MN (US); Robert E. Brennan, JR., River Falls, WI (US); Joseph J. Stoffel, Hastings, MN (US); Joseph P. Hensler, Woodbury, MN (US); Triet M. Lu, Woodbury, MN (US); Patrick A. Mach, Shorewood, MN (US); Chunmei Guo, Woodbury, MN (US); Mara S. Reif-Wenner, Red Wing, MN (US); Heather M. Webb, Woodbury, MN (US)

(21) Appl. No.: 12/743,518

(22) PCT Filed: Nov. 20, 2008
(86) PCT No.: PCT/US08/84186

§ 371 Date: Dec. 30, 2010

Related U.S. Application Data
(60) Provisional application No. 60/989,298, filed on Nov. 20, 2007.

(51) Int. Cl. G01N 33/53

(52) U.S. Cl. 435/7.2; 436/501

(57) ABSTRACT

The invention relates to methods of analyzing a sample for a bacterium of interest. In particular, the methods involve an initial capture process that includes the use of one or more antibodies having antigenic specificities for one or more distinct analytes characteristic of the specific bacterium. After initial capture of a specific bacterium, techniques of analyzing involve colorimetric techniques, particularly using colorimetric sensors that include polydiacetylene (PDA) materials.

### CROSS REFERENCE TO RELATED APPLICATIONS

This application claims the benefit of U.S. Provisional Patent Application No. 60/989,298, filed Nov. 20, 2007, which is incorporated herein by reference.

### GOVERNMENT RIGHTS

The U.S. Government may have certain rights to this invention under the terms of Contract No. DAAD-13-03-C-0047 (Program No. 2640) granted by the Department of Defense.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

One such microbe of significant interest is Staphylococcus aureus (“S. aureus”). This is a pathogen causing a wide spectrum of infections including: superficial lesions such as small skin abscesses and wound infections; systemic and life threatening conditions such as endocarditis, pneumonia, and septicemia; as well as toxinoses such as food poisoning and toxic shock syndrome. Some strains (e.g., Methicillin-Resistant S. aureus) are resistant to all but a few select antibiotics.

Current techniques for the detection of microbes, particularly bacteria resistant to antibiotics, are generally time consuming and typically involve culturing the bacteria in pure form. One such technique for the identification of pathogenic staphylococci associated with acute infection, i.e., S. aureus in humans and animals and S. intermedius and S. hyicus in animals, is based on the microbe's ability to clot plasma. At least two different coagulase tests have been described: a tube test for free coagulase and a slide test for “cell bound coagulase” or clumping factor. The tube coagulase test typically involves mixing an overnight culture in brain heart infusion broth with reconstituted plasma, incubating the mixture for 4 hours and observing the tube for clot formation by slowly tilting the tube. Incubation of the test overnight has been recommended for S. aureus since a small number of strains may require longer than 4 hours for clot formation. The slide coagulase test is typically faster and more economical; however, 10% to 15% of S. aureus strains may yield a negative result, which requires that the isolate by reexamined by the tube test.

Although methods of detecting S. aureus, as well as other microbes, have been described in the art, there would be advantage in improved methods of detection.

### SUMMARY

The invention provides methods of analyzing a sample for a bacterium of interest. In particular, the methods are useful for detecting one or more analytes characteristic of a bacterium of interest, such as components of cell walls that are characteristic of a bacterium, particularly Staphylococcus aureus.

The methods use a detection assay that includes a colorimetric sensor to detect the presence of analytes by spectral changes (color changes visible to the naked eye or with a colorimeter) that occur as a result of the interaction of the analyte(s) and/or probe(s), in a manner that causes conformational changes to diacetylene-containing polymer assemblies in a colorimetric sensor. A colorimetric sensor (i.e., sensor component) used in methods of the present invention preferably includes a polymerized composition that includes at least one diacetylene-containing polymer; a receptor incorporated in the polymerized composition to form a transducer; wherein the transducer exhibits a color change when contacted with the analyte(s) and/or probe(s). The detection assay typically also includes a buffer composition that mediates the interaction between the analyte(s) and the transducer.

In one embodiment, there is provided a method of analyzing a sample for a bacterium, the method including: providing a sample suspected of including one or more distinct analytes characteristic of a specific bacterium; providing one or more antibodies having antigenic specificities for the one or more distinct analytes (the analytes can be, for example, separate molecules like Protein A and Clumping Factor or two different epitopes of the same molecule) characteristic of the specific bacterium; providing a solid support material; providing contact between the sample, the solid support material, and the one or more antibodies under conditions effective to capture one or more analytes characteristic of a specific bacterium, if present; providing a colorimetric sensor that includes a polymerized composition including a diacetylene-containing polymer and a receptor, wherein the receptor is incorporated in the polymerized composition to form a transducer that provides a color change upon binding with one or more probe(s) and/or analyte(s); optionally removing the one or more analytes, if present, from the solid support material; and subsequent to capture and optional removal of the one or more analytes, subjecting the one or more analytes, if present, to direct or indirect analysis by the colorimetric sensor to analyze for the presence or absence of the specific bacterium (e.g., through the presence of one or more analytes or the absence of all analytes).

In certain embodiments, the one or more antibodies are attached to the solid support material forming an analyte-binding material, and the method includes providing contact between the sample and the analyte-binding material under conditions effective to capture one or more analytes characteristic of a specific bacterium, if present.

In certain embodiments, the analyte-binding material includes two or more antibodies having antigenic specificities for two or more distinct analytes characteristic of the specific bacterium. The two or more antibodies are preferably cooperative in their binding characteristics. That is, they are capable of simultaneously binding to distinct regions of the target analyte(s) or optionally are found to be of complementary binding whereby the binding of a distinct analyte is enhanced by the binding of another antibody.

The antibodies can be monoclonal, polyclonal, or combinations thereof. In certain embodiments, the antibodies are selected from the group consisting of MAb-76, MAb-107, affinity-purified RxClf40, affinity-purified GxClf40, MAb 12-9, fragments thereof, and combinations thereof.

In certain embodiments, the solid support material includes particulate material. Preferably, the particulate material includes magnetic particles.

In certain embodiments, the analyte-binding material includes particulate material that includes at least two portions, wherein one portion of particulate material has one antibody specific for one analyte disposed thereon, and a second portion has a different antibody specific for a distinct analyte disposed thereon. The two portions of particulate material may include the same types of particles. For example, particulate material can include at least two different types of particles, or the same type of particle, with two different antibodies attached to different particles.

Preferably, the one or more analytes characteristic of a specific bacterium are present on whole cells. Thus, certain methods of the present invention involve capturing whole bacterial cells.

Providing contact between the sample, the solid support material, and the one or more antibodies can include simultaneous and/or sequential (in any order desired), preferably simultaneous, contact between the sample, the solid support material, and the one or more antibodies.

In certain embodiments, the specific bacterium includes a Gram positive bacterium. A specific bacterium of particular interest includes Staphylococcus aureus.

In one embodiment, the present invention provides a method of analyzing a sample for a bacterium, the method that includes: providing a sample including whole cells suspected of including one or more distinct analytes characteristic of a specific bacterium; providing an analyte-binding material including magnetic particles, wherein the magnetic particles have disposed thereon one or more antibodies having antigenic specificities for one or more distinct analytes characteristic of the specific bacterium; providing a colorimetric sensor including a polymerized composition that includes at least one diacetylene-containing polymer and a receptor, wherein the receptor is incorporated in the polymerized composition to form a transducer that provides a color change upon binding with one or more probe(s) and/or analyte(s); providing contact between the sample and the analyte-binding material under conditions effective to capture the one or more analytes characteristic of a specific bacterium, if present on the whole cells; optionally removing the one or more analytes, if present, from the analyte-binding material; and subsequent to capture and optional removal of the one or more analytes, subjecting the one or more analytes, if present, to direct or indirect analysis by the colorimetric sensor to analyze for the presence or absence of the specific bacterium.

In another embodiment, the present invention provides a method of analyzing a sample for a Staphylococcus aureus bacterium, the method that includes: providing a sample including whole cells suspected of including one or more distinct analytes characteristic of a Staphylococcus aureus bacterium; providing an analyte-binding material including magnetic particles, wherein the magnetic particles have disposed thereon one or more antibodies having antigenic specificities for one or more distinct analytes characteristic of the Staphylococcus aureus bacterium; wherein the antibodies are selected from the group consisting of MAb-76, MAb-107, affinity-purified RxClf40, affinity-purified GxClf40, MAb 12-9, fragments thereof, and combinations thereof; providing a colorimetric sensor including a polymerized composition that includes at least one diacetylene-containing polymer and a receptor, wherein the receptor is incorporated in the polymerized composition to form a transducer that provides a color change upon binding with probe(s) and/or analyte(s); providing contact between the sample and the analyte-binding material under conditions effective to capture the one or more analytes characteristic of a Staphylococcus aureus bacterium, if present on the whole cells; optionally removing the one or more analytes, if present, from the analyte-binding material; and subsequent to capture and optional removal of the one or more analytes, subjecting the one or more analytes, if present, to direct or indirect analysis by the colorimetric sensor to analyze for the presence or absence of the Staphylococcus aureus bacterium.

In certain embodiments, the methods involve direct analysis and subjecting the one or more analytes, if present, to direct analysis by the colorimetric sensor includes providing contact between the one or more analytes and the colorimetric sensor.

In certain embodiments the methods involve indirect analysis, which involves the use of one or more probes. Preferably, such methods further include: providing one or more probes; providing conditions effective for the probes to bind to the one or more analytes, if present, before capture, after capture, or after optional removal from the solid support material; and providing contact between the unbound probes and the colorimetric sensor to analyze for the presence or absence of the specific bacterium.

“Whole cell” means a biologically active bacterial cell that retains its structure intact during separation from other biological materials, but does not necessarily need to be able to reproduce.

The terms “analyte” and “antigen” are used interchangeably and refer to various molecules (e.g., Protein A) or epitopes of molecules (e.g., different binding sites of Protein A), or whole cells or fragments of cells of the microorganism, that are characteristic of a microorganism (i.e., microbe) of interest. These include components of cell walls (e.g., cell-wall proteins such as protein A, and Clumping Factor, which is a cell wall-associated fibrinogen receptor that is found in S. aureus), external cell components (e.g., capsular polysaccharides and cell-wall carbohydrates), etc.

“Removing the one or more analytes from the analyte-binding material” means removing the various molecules, epitopes of molecules, whole cells, or fragments of cells that are characteristic of the microorganism of interest.

“Providing contact between the unbound probes and the colorimetric sensor” means providing contact between the probes, but it does not necessarily require direct contact between a specific analyte binding site (e.g., binding site of an antibody on the analyte), for example, and the colorimetric sensor.

“Providing contact between the one or more analytes and the colorimetric sensor” means providing contact between the various molecules, epitopes of molecules, whole cells, or fragments of cells that are characteristic of the microorganism of interest. This does not necessarily require direct contact between a specific analyte binding site (e.g., binding site of an antibody on the analyte), for example, and the colorimetric sensor.

“Magnetic particles” means particles or particle conglomerates comprised of ferromagnetic, paramagnetic, or superparamagnetic particles, including dispersions of said particles in a polymer bead.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “or” is generally employed in its sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 illustrates an embodiment of a sensor in solution in a test chamber.

FIG. 2 illustrates an embodiment of a sensor layer or portion on a substrate.

FIG. 3 illustrates a detection device having a sensor layer or portion and flow-through membrane where a body of the device is formed of a multiple layer construction.

### DETAILED DESCRIPTION OF ILLUSTRATIVE EMBODIMENTS

The present invention is directed to various methods of analyzing a sample for a bacterium of interest based on analysis of one or more analytes characteristic of the bacterium of interest. The methods of the present invention can involve not only detecting the presence of an analyte characteristic of the bacterium of interest, but preferably identifying such analyte, which can lead to identifying a bacterium for which the analyte is characteristic. In certain embodiments, analyzing the sample includes quantifying the analyte characteristic of the bacterium of interest.

The present invention provides a method for the detection of a specific analyte by combining a sample preparation system that captures target analyte(s) of interest with a detection assay using a colorimetric sensor. The sample preparation system includes material specific for capturing one or more analytes of interest, which may be present, for example, on one or more whole cells when captured.

Preferably, methods of the present invention involve an initial capture process that includes the use of one or more, and preferably two or more, antibodies having antigenic specificities for one or more, and preferably two or more, distinct analytes characteristic of the specific bacterium. If two or more antibodies are used, they are preferably cooperative in their binding characteristics. That is, they are capable of simultaneously binding to distinct regions of the target analyte(s) or optimally are found to be of complementary binding whereby the binding of a distinct analyte is enhanced by the binding of another antibody.

After initial capture of a specific bacterium, techniques of analyzing in methods of the present invention involve colorimetric techniques, particularly using colorimetric sensors that include polydiacetylene (PDA) materials. More specifically, the colorimetric sensor includes a polymerized composition including a receptor and a diacetylene-containing polymeric material (polydiacetylene assemblies), wherein the receptor is incorporated in the polymerized composition to form a transducer capable of providing a color change upon binding with one or more probe(s) and/or analyte(s).

The initial steps of an assay to detect one or more target analytes characteristic of a specific bacterium involves sample preparation including analyte capture. This preferably involves contacting an analyte-binding material with a sample suspected of containing the bacterium of interest (i.e., target bacterium), allowing the analyte-binding material to capture the analytes characteristic of the target bacterium (i.e., target analyte(s)). Alternatively, the sample of interest and separate components of the analyte-binding material (e.g., antibodies and magnetic particles) can be combined simultaneously.

A typical sample can include variable amounts of interfering substances. Interfering substances are other biological components and compounds that could interfere with the ability of the detection assay to sense the target analyte(s). A typical sample may also be eluted from a sample acquisition device, such as a swab, and as such could pick up interfering substances from the sample acquisition device that are not present in the original sample collected by that acquisition device.

A particularly preferred sample preparation system is one that includes an analyte-binding material and an elution buffer that preferentially capture the target analyte(s) while reducing (and preferably, eliminating) the capture (e.g., nonspecific capture) of potential interfering substances within the sample. A preferred analyte-binding material includes a magnetic solid support material, particularly magnetic particles. Upon completion of the capture step using such magnetic particles, a magnet is typically used to collect and concentrate the particles with analyte(s) attached thereto (i.e., the captured analyte(s)), allowing for the removal of the remainder of the sample containing potentially interfering substances. The particles with captured analyte(s) (i.e., particle-analyte complex) can then be resuspended in a clean buffer, if desired, to wash the particle-analyte complex of weakly bound contaminants. This washing process can be repeated several times if desired.

After analyte capture, the assay involves analysis using a colorimetric sensor. Preferably, the assay involves providing contact between the colorimetric sensor and the analyte-binding material with captured analyte (e.g., particle-analyte complex). Alternatively, the captured analyte can be removed from the analyte-binding material before it comes in contact with the colorimetric sensor.

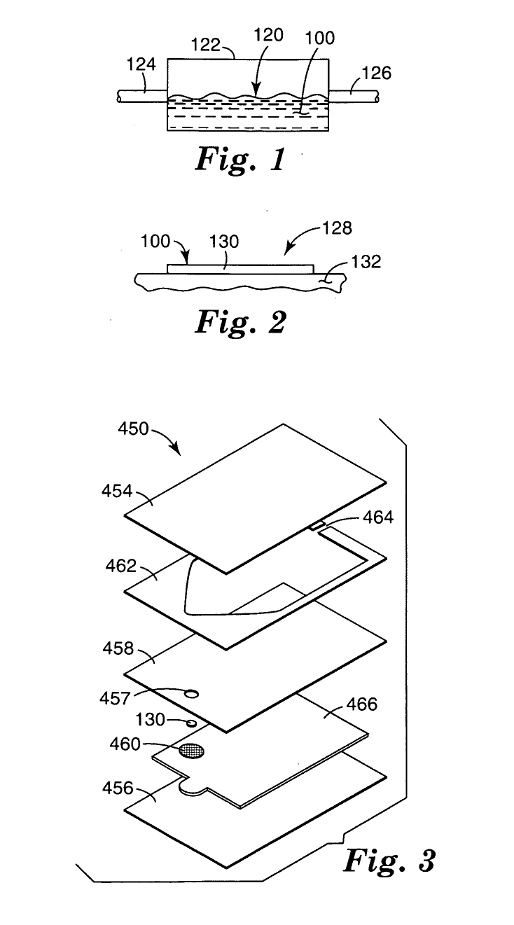

The colorimetric sensor can function in solution or be coated on a substrate. In general terms, as shown in FIG. 1, the sensor (i.e., sensor component) 100 is in solution 120 in a sensor chamber 122. As shown, the chamber 122 can be disposed in a flow path between a first flow path portion 124 and a second flow path portion 126. A test sample suspected of containing an analyte of interest flows into chamber 122 to mix with the solution 120. Upon mixing, the analyte of interest, if present in the test sample, binds with the receptor of the sensor component 100 to produce the detectable change.

FIG. 2 illustrates an exemplary embodiment wherein the sensor component 100 is formed of a sensor layer or portion 130 on a substrate 132, such as a thin film membrane, porous membrane, or other substrate. In one example, the sensor layer or portion 130 includes polydiacetylene liposomes deposited on a thin film membrane or other substrate.

In solution, the sensor can be used in a direct or an indirect (competitive) assay.

In a direct assay in solution, analyte-binding material with captured analyte (e.g., particle-analyte complex suspended in an appropriate buffer), or analyte after it has been captured and removed from the analyte-binding material, can directly bind to the colorimetric sensor producing a color change.

In an indirect assay in solution, one or more probes are first allowed to interact with the analyte-binding material having captured analyte attached thereto, or analyte after it has been captured and removed from the analyte-binding material, and subsequently unbound probe(s) bind to the colorimetric sensor producing a color change.

In a preferred method involving indirect analysis, a particle-analyte complex is suspended in an appropriate buffer, one or more probes are combined with such complex under conditions effective to form a particle-analyte-probe complex, which is separated from the liquid phase (e.g., by applying a magnetic field if the particles are magnetic), and the colorimetric sensor is introduced into the liquid phase under conditions that allow for unbound probe to bind to the colorimetric sensor producing a color change. In this indirect mode, the concentration of the unbound probe can be used to determine the concentration of captured analyte present originally. Although removal of the particle-analyte-probe complex before binding unbound probe to a colorimetric sensor is desired, this is not necessarily required because the particle-analyte-probe complex does not react with the colorimetric sensor with the same efficiency as unbound probe.

The color change resulting from an assay carried out in solution can be visually detected. Alternatively, if greater sensitivity is desired, an appropriate fluidic system can be used to concentrate the colorimetric sensor material onto a solid phase, thus amplifying the color change.

For colorimetric sensors coated on a substrate, analogous direct and indirect assays are also possible. In these assays, rather than placing the colorimetric sensor material in solution, the coated colorimetric sensor is exposed to the solution phase by employing an appropriate fluidic system, as disclosed in Applicants' Assignee's co-pending U.S. Patent Application Ser. No. 60/989,291, filed Nov. 20, 2007, entitled DETECTION DEVICES AND METHODS. A particularly preferred embodiment of such fluidic systems is illustrated herein in Examples 32-34 and FIG. 3, which illustrates a detection device having a sensor layer or portion and flow-through membrane where a body of the device is formed of a multiple layer construction.

Significantly, this initial capture of a specific bacterium, or analyte(s) characteristic thereof, allows for detection using a “universal” sensor system. It also allows for detection using a system capable of detecting multiple bacteria without requiring modification to tailor it to the target of interest. For example, a single transducer (polydiacetylene/receptor combination) could serve to detect a multitude of specific bacteria by combining it with a sample preparation specific to a given target bacteria.

Advantageously, methods of the invention can have improved sensitivity and specificity relative to other point-of-care tests such as lateral flow immunoassays. As further described in the examples presented herein, S. aureus can be detected at concentrations of 1×10⁵ colony forming units (“cfu”) per milliliter, 1×10⁴ cfu/mL, and 1×10³ cfu/mL. Accordingly, one of ordinary skill in the art appreciates that the methods of the present invention can be employed to detect target analytes at concentrations as low as 1×10³ cfu/mL. Target analytes can be detected at higher levels as well, ranging up to 5×10⁷ cfu/mL, for example.

Advantageously, methods of the invention can have improved overall detection times relative to other point-of-care tests such as lateral flow immunoassays and relative to culture methods. That is, methods of the invention can detect one or more analytes in a relatively short period of time. For example, S. aureus can be detected at any of the concentrations previously described in less than 60 minutes (e.g., 60 minutes, 30 minutes, 15 minutes, 10 minutes, or 5 minutes).

Using methods of the present invention, the capture time can be relatively short. For example, the capture time can be less than 30 minutes, less than 15 minutes, less than 5 minutes, less than 60 seconds, and even as short as 30 seconds. Such compositions may also include a buffer, such as phosphate buffered saline (PBS) optionally with a PLURONIC L-64 surfactant, ethylene diamine tetraacetic acid (EDTA), bovine serum albumin (BSA), or a combination thereof. Although physical agitation (or mixing) can be used for both large particles (e.g., having an average particle size or 1 micrometer (micron or μm) and small particles (e.g., having an average particle size of 200 nanometers (nm)), the small particles may be used without mixing.

Using methods of the present invention, the detection time can be relatively short. For example, the detection time can be less than 30 minutes, less than 15 minutes, less than 10 minutes, less than 5 minutes, and even as short as 1 minute.

Relatively small volumes of test sample can be used. Although test sample volume as high as 1-2 milliliters (mL) may be utilized, advantageously test samples on the order of 10 microliters (μL) are sufficient for methods of the present invention, with up to 200 μL being preferred for certain embodiments.

### Analyte-Binding Material: Reactant Molecules

The sample is contacted with appropriate reactant molecules for analyte binding (e.g., an analyte-binding material that includes a bacteria-recognizing reagent). Such reactant molecules include, for example, antibodies. Such antibodies can be attached to particulate material, a membrane, or other solid support material. Particularly preferred reactant molecules are those that are capable of direct interaction with target whole cells, particularly antibodies to whole cell surface antigens, and other proteins, such as Protein A, known to interact with whole cell surfaces.

Analyte-binding material useful in methods of the present invention for capture of the target analytes (e.g., target whole cells) typically includes a solid support material derivatized by coupling (non-covalently or covalently) to the support a reactant molecule that binds the target analytes. Preferably, a sample containing the target analytes (e.g., target whole cells) is contacted with the analyte-binding material to bind the target analytes, and unbound remaining mixture is removed from the support.

Bound analyte may be removed (e.g., eluted) from the support to obtain purified target analytes, or processed while attached to the analyte-binding material. This can be accomplished using wash buffers, for example, and varying pH and/or ionic strength. For example, certain derivatives of biotin such as 2-iminobiotin are available that bind to avidin in a pH sensitive manner. The sample and the beads are incubated between a pH of 9 and 11, at which pH avidin strongly interacts with 2-iminobiotin. Subsequent to capture, the target is eluted from the beads by changing the pH to 6 or lower, or by adding biotin (reducing the interaction between biotin and avidin).

As mentioned above, the target analytes on whole cells can be detected by a reactant molecule (e.g., an S. aureus reactant molecule or a bacteria-recognizing reagent for S. aureus). In some embodiments, one or more antibodies, such as an S. aureus antibody, are employed as a S. aureus reactant. “S. aureus antibody” refers to an immunoglobulin having the capacity to specifically bind a given antigen inclusive of antigen binding fragments thereof.

The term “antibody” is intended to include whole antibodies of a wide variety of isotypes (IgG, IgA, IgM, IgE, etc.), and fragments thereof from vertebrate, e.g., mammalian species which are also specifically reactive with foreign compounds, e.g., proteins.

The antibodies can be monoclonal, polyclonal, or combinations thereof. Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Non-limiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more binding sites. Antibodies can be labeled with a wide variety of detectable moieties known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

Various S. aureus antibodies are known in the art. For example, S. aureus antibodies are commercially available from Sigma-Aldrich and Accurate Chemical. Further, other S. aureus antibodies, such as the monoclonal antibody Mab 12-9, are described in U.S. Pat. No. 6,979,446. In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. Patent Application Publication No. 2008-0118937 and PCT Application No. US2007/084,736, both entitled “ANTIBODY WITH PROTEIN A SELECTIVITY,” and in U.S. patent application Ser. No. 11/562,747, filed on Nov. 22, 2006, and PCT Application Serial No. US2007/084,739, both entitled “ANTIBODY WITH PROTEIN A SELECTIVITY,” and in U.S. Patent Application Ser. No. 60/867,089, filed on Nov. 22, 2006 and U.S. patent application Ser. No. 11/943,168, filed Nov. 20, 2007, both of which are entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”).

More particularly, in one embodiment suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab 107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody MAb-76 to Protein A of S. aureus. The present invention can utilize monoclonal antibodies that bind to the same epitope of Protein A of S. aureus that is recognized by monoclonal antibody MAb-76. Methods for determining if a monoclonal antibody inhibits the binding of monoclonal antibody MAb-76 to Protein A of S. aureus and determining if a monoclonal antibody binds to the same epitope of Protein A of S. aureus that is recognized by monoclonal antibody MAb-76 are well known to those skilled in the art of immunology.

Suitable monoclonal antibodies are also those that inhibit the binding of monoclonal antibody MAb-107 to Protein A of S. aureus. The present invention can utilize monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also suitable for use in the present invention include various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibodies fragments produced by a Fab expression library, fragments including either a VL or VH domain, intracellularly-made antibodies (i.e., intrabodies), and antigen-binding antibody fragments thereof.

Monoclonal antibodies useful in the present invention may be of a wide variety of isotypes. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by a wide variety of methods known in the art for purification of an immunoglobulin molecule, for example, by chromatography (e.g., ion exchange, affinity, and sizing column chromatography), centrifugation, differential solubility, or by a wide variety of other standard techniques for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/mL), and more preferably up to 100 pg/mL. Suitable antibodies also include a high avidity anti-Staphylococcus aureus clumping factor protein polyclonal antibody preparation demonstrating at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to a S. aureus clumping factor (Clf40) protein affinity column; washing the column with a wash buffer having 0.5 M salt and a pH of 4; and eluting the high avidity anti-S. aureus clumping factor protein polyclonal antibody preparation from the column with an elution buffer with a pH of 2. Herein, the high avidity anti-Staphylococcus aureus clumping factor polyclonal antibody preparations from rabbits and goats are referred to as affinity-purified RxClf40 and affinity-purified GxClf40, respectively. In some embodiments, the high avidity anti-Staphylococcus aureus clumping factor protein polyclonal antibody preparation may be obtained by a method that further includes enriching the antiserum for the IgG class of antibodies prior to binding the antiserum to a S. aureus clumping factor (Clf40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing a wide variety of host animals. A wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

### Analyte-Binding Material: Solid Support Materials

Solid support materials can include particulate materials, membranes, gels (e.g., agarose), or other solid support materials such as the surfaces of tubes or plates. Exemplary solid support materials can include materials such as nitrocellulose, polystyrene, polypropylene, nylon, ferromagnetic materials, gold sols, polycarbonate, polyethylene, cellulose, polysaccharide, polyvinyl alcohol, or combinations thereof. For certain embodiments, particulate material and membranes are preferred.

Preferably, for certain embodiments, solid support material of the analyte-binding material includes functionalized particulate material (e.g., magnetic beads having an average particle size of less than 2 microns, and preferably, within a range of 0.05 micron to 1 micron). For example, magnetic beads functionalized with various groups such as carboxyl, amine, and tosyl are commercially available from Invitrogen (Carlsbad, Calif.) and Ademtech (Pessac, France). Streptavidin-coated particles are also available from several sources such as Invitrogen (Carlsbad, Calif.), Ademtech (Pessac, France), and Miltenyi Biotec GmbH (Bergisch Gladbach, Germany).

The analyte-binding material includes a solid support material, preferably particulate material, having one or more antibodies disposed on the solid support material. In certain embodiments, each particle of the particulate material has at least two antibodies that bind different analytes disposed thereon. For example, in certain embodiments, the analyte-binding material includes a solid support material (preferably particulate material) having antibodies MAb-76 and GxClfa disposed thereon (preferably, in a ratio of 1:1).

In certain embodiments, the analyte-binding material includes particulate material that includes at least two portions, wherein one portion of particulate material has one antibody specific for one analyte disposed thereon, and a second portion has a different antibody specific for a distinct analyte disposed thereon. The two portions of particulate material may include the same types of particles. Particulate material can include at least two different types of particles, or the same type of particle, with at least two different antibodies attached to different particles.

Antibodies can be attached to a support material, preferably a particulate support material, through either covalent attachment or non-covalent attachment.

Non-covalent attachment of an antibody to a solid support material includes attachment by ionic interaction or hydrogen bonding, for example. One example of a non-covalent attachment included in the present invention is the well-know biotin-avidin system. Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in “Applications of Avidin-Biotin Technology to Affinity-Based Separation,” Bayer et al., J. of Chromatography, pgs. 3-11 (1990).

Streptavidin, and its functional homolog avidin, are tetrameric proteins, having four identical subunits. Streptavidin is secreted by the actinobacterium Streptomyces avidinii. A monomer of streptavidin or avidin contains one high-affinity binding site for the water-soluble vitamin biotin and a streptavidin or avidin tetramer binds four biotin molecules.

Biotin, also known as vitamin H or cis-hexahydro-2-oxo-1H-thieno-[3-,4]-imidazole-4-pentanoic acid, is a basic vitamin which is essential for most organisms including bacteria and yeast. Biotin has a molecular weight of about 244 daltons, much lower than its binding partners avidin and streptavidin. Biotin is also an enzyme cofactor of pyruvate carboxylase, trans-carboxylase, acetyl-CoA-carboxylase and beta-methylcrotonyl-CoA carboxylase which together carboxylate a wide variety of substrates.

Both streptavidin and avidin exhibit extremely tight and highly specific binding to biotin which is one of the strongest known non-covalent interactions between proteins and ligands, with a molar dissociation constant of 10⁻¹⁵ molar (M) (Green, Advances in Protein Chemistry, Vol. 29, pp. 85-133 (1975)), and a t½ of ligand dissociation of 89 days (Green, Advances in Protein Chemistry, Vol. 29, pp. 85-133 (1975)). The avidin-biotin bond is stable in serum and in the circulation (Wei et al., Experientia, Vol. 27, pp. 366-368 (1970)). Once formed, the avidin-biotin complex is unaffected by most extremes of pH, organic solvents and denaturing conditions. Separation of streptavidin from biotin requires conditions, such as 8M guanidine, pH 1.5, or autoclaving at 121° C. for 10 minutes (min).

Antibodies may be biotinylated using a wide variety of known methodologies. For example, antibodies may be biotinylated chemically, using activated biotin analogues, such as N-hydroxysuccinimidobiotin (NHS-biotin), which is commercially available from Pierce Chemical Company, Rockford, Ill., and requires the presence of a free primary amino group on the antibody.

In a preferred method of the present invention, magnetic particles can be coated with streptavidin and contacted with biotinylated antibodies. These particles can then be used for bacterial capture. With two or more antibodies, simultaneous or sequential capture can occur. Sequential capture can involve a wide variety of reagent orders of addition, as would be understood by one of skill in the art.

In another method biotinylated antibodies may be mixed with the sample to capture the bacteria first, subsequently the biotinylated antibody-bacteria complex can then be non-covalently bound to the straptavidin coated bead. For certain embodiments, the ratio of biotin to antibody can be optimized to avoid aggregation for certain particles.

For certain embodiments, the ratio of the number of biotin molecules to the number of antibodies can be optimized to avoid aggregation for certain particles. For example, with the Ademtech 200 nm streptavidin coated particles, a ratio of around 2:1 is preferred. Higher ratios, especially greater than 7:1 have shown aggregation issues for these particles.

Representative methods for covalent attaching an antibody to a particulate support material include utilizing functional groups in the support materials (such as carboxyl, amine, hydroxyl, maleimide, hydrazide) activated by activation compounds (such as glutaraldehyde, carbodiimide, cyanogens bromide) to react with another reactive groups (such as hydroxyl, amino, amido, or sulfhydryl groups) in an antibody. This bond may be, for example, a disulfide bond, thioester bond, amide bond, thioether bond, and the like. Antibodies may also be directly attached to support material functionalized with groups (such as tosyl, chloromethyl) that can directly react with a functional group on the antibody (such as amine).

Antibodies may be covalently bonded to a particulate support material by a wide variety of the methods known in the art. For example, beads derivatized with carboxyl groups are commercially available. Antibodies can then be coupled to these beads through the formation of an amide linkage between a primary amine on the antibody and the carboxyl groups on the bead surface. The coupling reaction is mediated by activation via carbodiimide.

Typically, the particle concentration and antibody-to-particle ratios are optimized for the system of interest to achieve rapid capture. Generally, this is particle dependent. For example, for Dynal 1-micron (μm) particles the particle concentration is preferably greater than 0.04 milligrams per milliliter (mg/mL), more preferably greater than 0.1 mg/mL, and even more preferably greater than 0.16 mg/mL. For the same particles, the antibody to particle ratio is preferably greater than 1 μg of antibody per 1 mg of particles, more preferably greater than 10 μg/mg, and even more preferably greater than 40 μg/mg particles.

In another embodiment of the capture step of the method of this invention, when employing sub-micron size particles (e.g., capture beads), a particle concentration is preferably greater than 0.04 mg/mL, more preferably greater than 0.1 mg/mL, and even more preferably greater than 0.16 mg/mL. In another embodiment of the capture step of the method of this invention, when employing sub-micron size particles, the antibody to particle ratio is preferably greater than 0.01 μg of antibody per 1 mg of particles, more preferably greater than 0.1 μg of antibody per 1 mg of particles, and even more preferably greater than 10 μg of antibody per 1 mg of particles, but typically not exceeding 10 μg of antibody per 1 mg of particles.

Suitable particles may or may not be blocked to prevent nonspecific binding. Such blocking may be done before or after antibody attachment. For example, certain magnetic beads (e.g., Dynal T1 MyOne streptavidin beads) are purchased blocked with bovine serum albumin (BSA). Other suitable blocking agents for nonspecific binding may be used, as is well known in the art. Also, a blocking agent (e.g., a polymyxin) can be used to prevent nonspecific binding of probes (e.g., a polymyxin) in the colorimetric sensor.

Particles may be separated from the sample by settling, centrifugation, or filtration. Preferably, magnetic particles are used and they are separated by the use of a magnetic field. Such separated particles (preferably having whole cells thereon) can be washed with various buffers including, for example, PBS with PLURONIC L-64, or TWEEN 20, with or without BSA, etc.

Significantly, using methods of the present invention, preferably at least 20% of the target whole cells are captured, more preferably at least 50% of the target whole cells are captured, and even more preferably at least 80% of the target whole cells are captured.

### Colorimetric Sensor: Polydiacetylene Assemblies

Colorimetric sensors suitable for use in methods of the present invention include a polymerized composition including a receptor and a diacetylene-containing polymeric material (polydiacetylene assemblies), wherein the receptor is incorporated in the polymerized composition to form a transducer capable of providing a color change upon binding with one or more probe(s) and/or analyte(s). Such colorimetric sensors can serve as the basis for the colorimetric detection of a molecular recognition event.

Suitable diacetylene compounds for use in colorimetric sensors self assemble in solution to form ordered assemblies that can be polymerized using actinic radiation such as, for example, electromagnetic radiation in the UV or visible range of the electromagnetic spectrum. Polymerization of the diacetylene compounds result in polymerization reaction products that have a color in the visible spectrum less than 570 nanometers (nm), between 570 nm and 600 nm, or greater than 600 nm, depending on their conformation and exposure to external factors. Typically, polymerization of the diacetylene compounds disclosed herein result in meta-stable blue phase polymer networks that include a polydiacetylene backbone. These meta-stable blue phase polymer networks undergo a color change from bluish to reddish-orange upon exposure to external factors such as heat, a change in solvent or counterion, if available, or physical stress, for example.

The ability of the diacetylene compounds and their polymerization products disclosed herein to undergo a visible color change upon exposure to physical stress make them candidates for the preparation of sensing devices for detection of an analyte. The polydiacetylene assemblies formed from the disclosed diacetylene compounds can function as a transducer in biosensing applications.

The structural requirements of a diacetylenic molecule for a given sensing application are typically application specific. Features such as overall chain length, solubility, polarity, crystallinity, and presence of functional groups for further molecular modification all cooperatively determine a diacetylenic molecule's ability to serve as a useful sensing material.

For example, in the case of biodetection of an analyte in aqueous media, the structure of the diacetylenic compound should be capable of forming a stable dispersion in water, polymerizing efficiently to a colored material, incorporating appropriate receptor chemistry for binding to an analyte, and transducing that binding interaction by means of a color change. These abilities are dependent on the structural features of the diacetylene compounds.

The diacetylene compounds of the present invention possess the capabilities described above and can be easily and efficiently polymerized into polydiacetylene assemblies that undergo the desired color changes. Additionally, the diacetylene compounds allow for the incorporation of large excesses of unpolymerizable material, such as a receptor described below, while still forming a stable, polymerizable solution.

The disclosed diacetylene compounds can be synthesized in a rapid high-yielding fashion, including high-throughput methods of synthesis. The presence of functionality in the backbones of the diacetylenic compounds, such as heteroatoms for example, provides for the possibility of easy structural elaboration in order to meet the requirements of a given sensing application. The diacetylenic compounds can be polymerized into the desired polydiacetylene backbone containing network by adding the diacetylene to a suitable solvent, such as water for example, sonicating the mixture, and then irradiating the solution with ultraviolet light, typically at a wavelength of 254 nm. Upon polymerization the solution undergoes a color change to bluish-purple.

Diacetylenes useful in the present invention typically contain an average carbon chain length of 8 with at least one functional group such as a carboxyl group, primary and tertiaty amine groups, methyl esters of carboxyl, etc. Suitable diacetylenes include those described in U.S. Pat. No. 5,491,097 (Ribi et al.); PCT Publication No. WO 02/00920; U.S. Pat. No. 6,306,598 and PCT Publication WO 01/71317.

In a preferred embodiment, the polydiacetylene assemblies are polymerized compounds of the formula

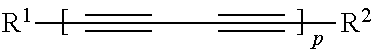

where R¹ is
alkyl,

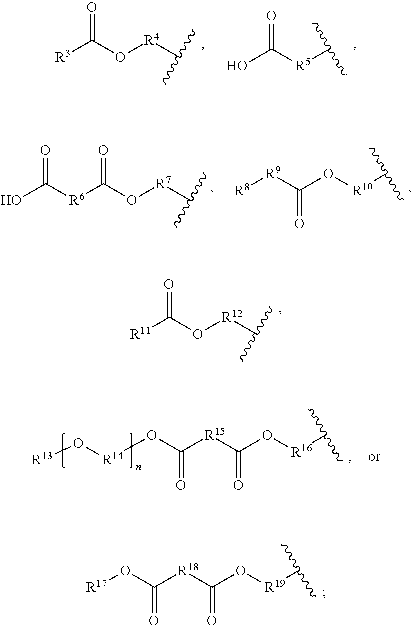

R² is

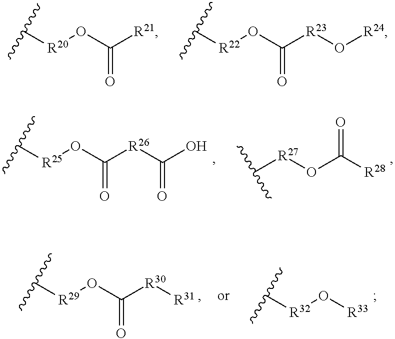

R³, R⁸, R¹³, R²¹, R²⁴, R³¹ and R³³ are independently alkyl; R⁴, R⁵, R⁷, R¹⁴, R¹⁶, R¹⁹, R²⁰, R²², R²⁵, and R³² are independently alkylene; R⁶, R¹⁵, R¹⁸, and R²⁶ are independently alkylene, alkenylene, or arylene; R⁹ is alkylene or —NR³⁴—; R¹⁰, R¹², R²⁷, and R²⁹ are independently alkylene or alkylene-arylene; R¹¹ and R²⁸ are independently alkynyl; R¹⁷ is an ester-activating group; R²³ is arylene; R³⁰ is alkylene or —NR³⁶—; R³⁴, and R³⁶ are independently H or C₁-C₄ alkyl; p is 1-5; and n is 1-20; and where R¹ and R² are not the same. Exemplary compounds are further described in U.S. Pat. No. 6,963,007 and U.S. patent application Publication Ser. Nos. 04-0126897-A1 and 04-0132217-A1. In a preferred embodiment, R¹ is

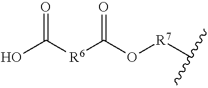

wherein R⁷ is ethylene, trimethylene, tetramethylene, pentamethylene, hexamethylene, heptamethylene, octamethylene, or nonamethylene, and R⁶ is ethylene, trimethylene, ethenylene, or phenylene; and wherein R² is

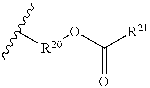

wherein R²⁰ is ethylene, trimethylene, tetramethylene, pentamethylene, hexamethylene, heptamethylene, octamethylene, or nonamethylene, and wherein R²¹ is undecyl, tridecyl, pentadecyl, heptadecyl; and wherein p is 1.

The invention is inclusive of the compounds described herein including isomers, such as structural isomers and geometric isomers, salts, solvates, polymorphs and the like.

Diacetylenes of the Formula XXIII can be prepared as outlined in Scheme 1 where n is typically 1 to 4 and m is typically 10 to 14.

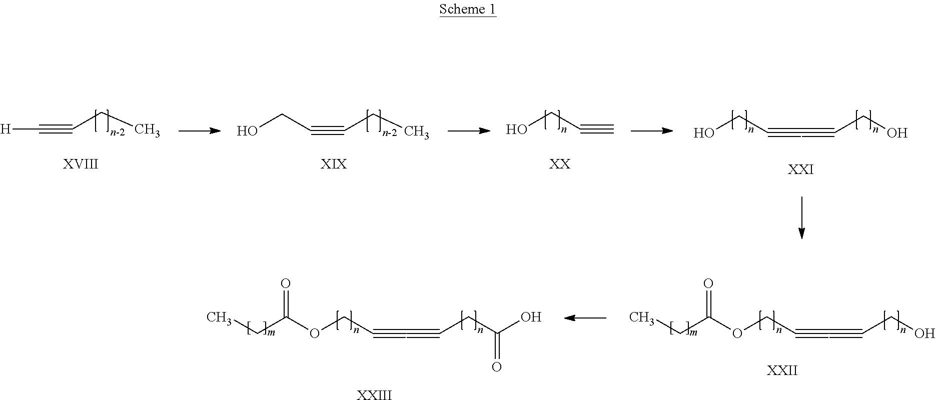

Compounds of formula XXIII can be prepared via oxidation from compounds of formula XXII by reaction with a suitable oxidizing agent in a suitable solvent such as DMF for example. Suitable oxidizing agents include Jones reagent and pyridinium dichromate for example. The aforesaid reaction is typically run for a period of time from 1 hour to 48 hours, generally 8 hours, at a temperature from 0° C. to 40° C., generally from 0° C. to 25° C.

Compounds of formula XXII can be prepared from compounds of formula XXI by reaction with a suitable acid chloride. Suitable acid chlorides include an acid chloride that affords the desired product such as lauroyl chloride, 1-dodecanoyl chloride, 1-tetradecanoyl chloride, 1-hexadecanoyl chloride, and 1-octadecanoyl chloride for example. Suitable solvents include ether, tetrahydrofuran, dichloromethane, and chloroform, for example. The aforesaid reaction is typically run for a period of time from 1 hour to 24 hours, generally 3 hours, at a temperature from 0° C. to 40° C., generally from 0° C. to 25° C., in the presence of a base such as trialkylamine or pyridine base.

Compounds of formula XXI are either commercially available (e.g., where n is 1-4) or can be prepared from compounds of the formula XVIII via compounds XIX and XX as outlined in Scheme 1 and disclosed in Abrams et al., Org. Synth., 66, 127-31 (1988) and Brandsma, Preparative Acetylenic Chemistry, (Elsevier Pub. Co., New York, 1971), for example.

Diacetylenic compounds as disclosed herein can also be prepared by reacting compounds of formula XXII with an anhydride such as succinic, glutaric, or phthalic anhydride in the presence of a suitable solvent such as toluene. The aforesaid reaction is typically run for a period of time from 1 hour to 24 hours, generally 15 hours, at a temperature from 50° C. to 125° C., generally from 100° C. to 125° C.

A sensor comprising the polydiacetylene assemblies can be obtained without the need to form a film by the conventional LB (Langmuir-Blodgett) process before transferring it onto an appropriate support. Alternatively, the polydiacetylene assemblies can be formed on a substrate using the known LB process as described in A. Ulman, An Introduction to Ultrathin Organic Films, Academic Press, New York, pp. 101-219 (1991).

### Colorimetric Sensor: Receptors

The colorimetric sensor includes a transducer formed from a receptor incorporated within the polydiacetylene assemblies in solution. The sensor can be prepared by adding a receptor to the diacetylene monomers either prior to or after polymerization. The receptor is capable of functionalizing the polydiacetylene assemblies through a variety of means including physical mixing, covalent bonding, and non-covalent interactions (such as electrostatic interactions, polar interactions, etc).

Upon polymerization or thereafter, the receptor is effectively incorporated with the polymer network such that interaction of the receptor with an analyte or probe results in a visible color change due to the perturbation of the conjugated ene-yne polymer backbone.

The incorporation of the receptor with the polydiacetylene assembly provides a structural shape capable of deformation in response to interaction or binding with one or more probes and/or analytes. Particularly useful receptors are assemblies of amphiphilic molecules with typically a rod shape molecular architecture that can be characterized by a packing parameter defined as: v/(a₀1_{c}) (Israelachvili et al., Q. Rev. Biophys., 13, 121 (1980)), where v is the volume taken up by the hydrocarbon components of the molecules (for example, the hydrocarbon chains of a phospholipid or a fatty acid), a₀ is the effective area taken up by the polar headgroup (for example the phosphate headgroup of a phospholipid or the carboxylic acid headgroup of a fatty acid), and 1_{c} is the so-called critical length, and generally describes the length of the molecule at the temperature of its environment. Preferred amphiphilic molecules for a receptor are those with packing parameters v/(a₀1_{c}) values between ⅓ and 1.

Examples of useful receptors include, but are not limited to, lipids, surface membrane proteins, enzymes, lectins, antibodies, recombinant proteins, etc.; synthetic proteins; nucleic acids; c-glycosides; carbohydrates; gangliosides; and chelating agents. In most embodiments, the receptor is a phospholipid. Suitable phospholipids include phosphocholines (e.g., 1,2-dimeristoyl-sn-glycero-3-phosphocholine,); phosphoethanolamines; and phosphatidylethanolamines; phosphatidylserines; and phosphatidylglycerols such as those described in Silver, The Physical Chemistry of Membranes, Chapter 1, pp 1-24 (1985).

In one embodiment, the receptor is physically mixed and dispersed among the polydiacetylene to form a structure wherein the structure itself has a binding affinity for the probes and/or analytes of interest. Structures include, but are not limited to, liposomes, micelles, and lamellas. In a preferred embodiment, the structure is a liposome. While not intending to be bound by theory, it is believed that the phospholipid mimics a cell membrane while the polydiacetylene assemblies allow the physico-chemical changes occurring to the liposomes to be translated into a visible color change. The liposomes as prepared possess a well-defined morphology, size distribution and other physical characteristics such as a well-defined surface potential.

The ratio of receptor to diacetylene compounds in the liposome can be varied based on the selection of materials and the desired colorimetric response. In most embodiments, the ratio of phospholipids to diacetylene compound will be at least 25:75, and more preferably at least 40:60. In a preferred embodiment, the liposomes are composed of the diacetylene compound: HO(O)C(CH₂)₂C(O)O(CH₂)₄C≡C—C≡C(CH₂)₄O(O)C(CH₂)₁₂CH₃ [succinic acid mono-(12-tetradecanoyloxy-dodeca-5,7-diynyl)ester], and the zwitterionic phospholipid 1,2-dimeristoyl-sn-glycero-3-phosphocholine [DMPC] mixed in a 6:4 ratio.

The liposomes can be prepared by probe sonication of the material mixture suspended in a buffer solution that is referred to as the preparation buffer. For example, the preparation buffer can be a low ionic strength (5 mM) N-2-Hydroxyethylpiperazine-N′-2-ethanesulfonic acid [HEPES] buffer (pH=7.2). Another useful preparation buffer is a low ionic strength (2 mM) Tris Hydroxymethylaminoethane [TRIS] buffer (pH=8.5).

### Colorimetric Sensor: Probes

The colorimetric sensor of the present invention is preferably designed to exploit the way one or more probes can interact with liposomes containing both a receptor, such as phospholipids, and polymerized diacetylenes. The liposomes can be thought as models for biological membranes and their interaction with probes, such as a protein, can be described as in Oellerich et al., J. Phys. Chem. B, 108, 3871-3878 (2004); and Zuckermann et al., Biophysi. J., 81, 2458-2472 (2001).

It is convenient to describe the interaction of proteins with liposomes in terms of the lipid (partitioned in the liposome phase) to protein concentration ratio. At high lipid to protein concentration ratios, proteins will adsorb to the surface of the liposomes primarily through electrostatic interactions. As the protein concentration is increased, and the lipid to protein concentration ratio is lowered, proteins continue to adsorb electrostatically to the surface of a liposome until they completely saturate or envelop the liposomes. As this process proceeds, both liposomes and the proteins can undergo morphological and conformational changes, until the hydrophobic segment of the proteins covering the liposome surface can begin to interact with the hydrophobic interior of the liposome structure. At this point, the proteins can become hydrophobically bound and penetrate the liposome structure, resulting in substantial morphological change in the liposome structure, with the size and permeability of the liposomes changing drastically. Eventually, the layers of adsorbed proteins can result in the loss of suspension stability, via flocculation of the liposomes, and finally, precipitation of the lipid phase.

The presence of these electrostatic interactions is highly dependent not only on the type of proteins and lipids present but on their environment as well. Although not desiring to be bound by theory, it is believed that the ionic strength of a given buffer composition would be helpful in establishing the surface potential of both liposomes and charged proteins, and thus their ability to interact significantly electrostatically.

For example, in a buffer composition of low ionic strength (2-5 mM) at neutral pH (e.g., HEPES, TRIS), a charged probe can electrostatically adsorb to the polydiacetylene liposomes. Although the initial adsorption may not in itself trigger a substantial change in the size and morphology of the liposome, and thus an initially small or negligible colorimetric response, if the probe is present in excess to the lipid, it is likely that the probe will eventually become hydrophobically bound to the liposome and penetrate its interior membrane structure. At this point, one would expect that the large mechanical stresses imparted by the incorporation of the probe within the liposome structure would significantly change the polydiacetylene conformation, resulting in a concomitant colorimetric response readily observable.

Alternatively, if the probe is negatively charged at neutral pH its capacity to interact electrostatically with the polydiacetylene liposomes is severely hindered, and the ability to generate a colorimetric response due to a hydrophobic interaction between probe and the receptor-containing polydiacetylene liposomes may be compromised. In this event, using a high ionic strength buffer (greater than 100 millimolar (mM)) at neutral pH (e.g., phosphate buffer saline PBS, Imidazole buffer) would provide a mean to decrease the surface potential of the liposomes (by screening the surface charge of the liposome), facilitating the direct hydrophobic interaction of non-charged probes with the liposomes, and resulting in the incorporation of that protein within the structure of the liposome. Thus, in this case, the buffer composition assists in enabling a substantial colorimetric response, which would otherwise not take place. Although the higher ionic strength of the buffer composition, because of its effect on the surface potential of the liposomes, can introduce a significant colorimetric response in the absence of a probe, we have determined that when the probe is present, the colorimetric response is significantly enhanced due to the protein-liposome hydrophobic interactions. This result has very useful practical consequences: the detection time at a given limit of detection can be significantly shortened, or conversely, for a fixed assay time the limit of detection can be significantly lowered.

Based on this phenomena, the probe can be selected based on its ability to interact specifically with both a given analyte target and the polydiacetylene liposome to trigger a colorimetric response. The colorimetric response of the polydiacteylene-containing liposome is directly proportional to the concentration of the probe or a probe-analyte complex.

The selection of probe(s) for a particular application will depend in part on the probe's size, shape, charge, hydrophobicity and affinity towards molecules. The probes may be positively charged, negatively charged, or zwitterionic depending on the pH of the environment. At a pH below the isoelectric point of a probe, the probe is positively charged and above this point it is negatively charged. As used herein, the term “isoelectric point” refers to the pH at which the probe has a net charge of zero.

In order to design a biochemical assay with a polydiacetylene/phospholipid system, knowing the isoelectric point of the receptor (or probe) will affect the choice of buffer combinations. A probe with lower isoelectric point may require higher ionic strength buffers to obtain a change in morphology of the liposome. A higher isoelectric point protein can be used in low ionic strength buffer like HEPES buffer to produce a color change.

The probes can be a molecule with an affinity for both the target analyte and the receptor. Possible probes for use in the present invention include membrane disrupting peptides such as alamethicin, magainin, gramicidin, polymyxin B sulfate, and melittin; fibrinogen; streptpyridin; antibodies; lectins; and combinations thereof. See, e.g., U.S. Patent Application Publication No. 2004/132217. A polymyxin, such as polymyxin B sulfate, is particularly useful for detecting Gram positive bacteria.

Antibodies and antibody fragments can also be employed as the probe. This includes segments of proteolytically cleaved or recombinantly prepared portions of an antibody molecule that are capable of selectively reacting with a certain protein. Nonlimiting examples of such proteolytic and/or recombinant fragments include F(ab′), F(ab)₂, Fv, and single chain antibodies (scFv) containing a VL and/or VH domain joined by a peptide linker. The scFv's can be covalently or non-covalently linked to form antibodies having two or more binding sites. The scFv's can be covalently or non-covalently linked to form antibodies having two or more binding sites. Antibodies can be labeled with a wide variety of detectable moieties known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

Various S. aureus antibodies are known in the art as described herein above in the context of analyte-binding material.

### Detection Assay Buffer Compositions

The detection assay typically also includes a buffer composition that mediates the interaction between the analyte(s) and the transducer. The buffer composition provides a system capable of resisting changes in pH in the presence of other components, consisting of a conjugate acid-base pair in which the ratio of proton acceptor to proton donor is near unity. In addition, the buffer compositions of the present invention mediate the physical or chemical interaction between the analyte and the components of the colorimetric sensor. For example, appropriate choice of the buffer composition can facilitate the interaction of a protein probe with the diacetylene liposomes, while inhibiting the interaction of other potentially interfering proteins that may be present in the sample. Buffer compositions that may be particularly useful include HEPES buffer, Imidazole buffer, and PBS buffer.

For example, in a system containing only HEPES buffer, which has a pH of 7.2, polymyxin B sulfate (with an isoelectric point of 7.7) has a positive charge and readily adheres to the negatively charged polar head group of a phospholipids, and can induce a color change from blue to red in the colorimetric sensor. See, e.g., U.S. Patent Application Publication No. 2006/134796. Furthermore, Human Serum Albumin, an abudant protein in wound exudate, with an isoelectric point typically in a range of 4.5 to 5.5, has a negative charge in the same HEPES buffer composition, minimizing adsorption or electrostatic interactions with the polar head group of the phospholipids and mititgating the potential for interference with the assay.

Alternatively, in the presence of the buffers with higher ionic strength, such as imidazole or PBS, the ionic strength alters the morphology of the liposome (or other transducer structure) to expose the hydrophobic portions, thus allowing direct interactions with the hydrophobic portion of a protein to cause a color change.

Finally, in an analogous manner, one could introduce a surfactant component in the buffer composition that can assist the hydrophobic interaction of a probe with the colorimetric sensor. Surfactants that may be particularly useful in the present invention include nonionic surfactants. Polyalkoxylated, and in particular polyethoxylated, nonionic surfactants can stabilize the components of the present invention in solutions particularly well.

Surfactants of the nonionic type that may be useful include:

1. Polyethylene oxide extended sorbitan monoalkylates (i.e., Polysorbates). In particular, a Polysorbate 20 commercially available as NIKKOL TL-10 (from Barret Products) is very effective.

2. Polyalkoxylated alkanols. Surfactants such as those commercially available under the trade designation BRIJ from ICI Specialty Chemicals, Wilmington, Del. having an HLB of at least about 14 have proven useful. In particular, BRIJ 78 and BRIJ 700, which are stearyl alcohol ethoxylates having 20 and 100 moles of polyethylene oxide, respectively, have proven very useful. Also useful is a ceteareth 55, which is commercially available under the trade designation PLURAFAC A-39 from BASF Corp., Performance Chemicals Div., Mt. Olive, N.J.

3. Polyalkoxylated alkylphenols. Useful surfactants of this type include polyethoxylated octyl or nonyl phenols having HLB values of at least about 14, which are commercially available under the trade designations ICONOL and TRITON, from BASF Corp., Performance Chemicals Div., Mt. Olive, N.J. and Union Carbide Corp., Danbury, Conn., respectively. Examples include TRITON X100 (an octyl phenol having 15 moles of ethylene oxide available from Union Carbide Corp., Danbury, Conn.) and ICONOL NP70 and NP40 (nonyl phenol having 40 and 70 moles of ethylene oxide units, respectively, available from BASF Corp., Performance Chemicals Div., Mt. Olive, N.J.). Sulfated and phosphated derivatives of these surfactants are also useful. Examples of such derivatives include ammonium nonoxynol-4-sulfate, which is commercially available under the trade designation RHODAPEX CO-436 from Rhodia, Dayton, N.J.

4. Polaxamers. Surfactants based on block copolymers of ethylene oxide (EO) and propylene oxide (PO) have been shown to be effective at stabilizing the film-forming polymers of the present invention and provide good wetting. Both EO-PO-EO blocks and PO-EO-PO blocks are expected to work well as long as the HLB is at least about 14, and preferably at least about 16. Such surfactants are commercially available under the trade designations PLURONIC and TETRONIC from BASF Corp., Performance Chemicals Div., Mt. Olive, N.J. It is noted that the PLURONIC surfactants from BASF have reported HLB values that are calculated differently than described above. In such situation, the HLB values reported by BASF should be used. For example, preferred PLURONIC surfactants are L-64 and F-127, which have HLBs of 15 and 22, respectively. Although the PLURONIC surfactants are quite effective at stabilizing the compositions of the present invention and are quite effective with iodine as the active agent, they may reduce the antimicrobial activity of compositions using povidone-iodine as the active agent.

5. Polyalkoxylated esters. Polyalkoxylated glycols such as ethylene glycol, propylene glycol, glycerol, and the like may be partially or completely esterified, i.e., one or more alcohols may be esterified, with a (C8-C22) alkyl carboxylic acid. Such polyethoxylated esters having an HLB of at least about 14, and preferably at least about 16, are suitable for use in compositions of the present invention.

6. Alkyl Polyglucosides. Alkyl polyglucosides, such as those described in U.S. Pat. No. 5,951,993 (Scholz et al.), starting at column 9, line 44, are compatible with the film-forming polymers of the present invention and may contribute to polymer stability. Examples include glucopon 425, which has a (C8-C16)alkyl chain length with an average chain length of 10.3 carbons and 1-4 glucose units.

### Samples and Analytes

Bacteria of particular interest include Gram positive and Gram negative bacteria. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli O157 and multiple drug resistant Gram negative rods (MDR).

Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Species of interest can be analyzed in a test sample that may be derived from a wide variety of sources, such as a physiological fluid, e.g., blood, saliva, ocular lens fluid, synovial fluid, cerebral spinal fluid, pus, sweat, exudate, urine, mucus, mucosal tissue (e.g., buccal, buccal, gingival, nasal, ocular, tracheal, bronchial, gastrointestinal, rectal, urethral, ureteral, vaginal, cervical, and uterine mucosal membranes), lactation milk, or the like. Further, the test sample may be derived from a body site, e.g., wound, skin, nares, nasopharyngeal cavity, nasal cavities, anterior nasal vestibule scalp, nails, outer ear, middle ear, mouth, rectum, vagina, or other similar site. Besides physiological fluids, other test samples may include other liquids as well as solid(s) dissolved in a liquid medium. Samples of interest may include process streams, water, soil, plants or other vegetation, air, (e.g., contaminated) surfaces, and the like.

The art describes various patient sampling techniques for the detection of bacteria, such as S. aureus. Such sampling techniques are suitable for the methods of the present invention as well. For example, it is common to obtain a sample from wiping the nares of a patient, e.g., patient's anterior nares, by swabbing with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample collection devices are commercially available, for example, from Puritan Medical Products Co. LLC, Guilford, Me., under the trade designation PURE-WRAPS, or from Copan Diagnostics, Inc., Murrietta, Calif., under the trade designations microRheologics nylon flocked swab and ESwab Collection and Transport System. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

In certain embodiments, the sample of material is typically eluted (or “released” or “washed”) from the sample collection device using a buffer solution such as by example, water, physiological saline, pH buffered solutions, or any other solutions or combinations of solutions that elute an analyte or sample from the sample acquisition device. An example of an elution buffer includes, for example, phosphate buffered saline (PBS), which can be used in combination, for example, with TWEEN 20 (polyoxyethylene sorbitan monolaurate, available from Sigma-Aldrich Corp.) or PLURONIC L64 (poly(oxyethylene-co-oxypropylene) block copolymer, available from BASF Corp.). Other extraction solutions function to maintain specimen stability during transport from sample collection site to sample analysis sites. Examples of these types of extraction solutions include Amies' and Stuart's transport media.

The test sample (e.g., liquid) may be subjected to prior treatment, such as dilution of viscous fluids. The test sample (e.g., liquid) may be subjected to other methods of treatment prior to injection into the sample port such as concentration, by filtration, distillation, dialysis, dilution, inactivation of natural components, addition of reagents, chemical treatment, etc.

That is, the test sample can be prepared using a wide variety of means well-known to those of skill in the art. For example, the sample could be disrupted to make available for analysis an analyte characteristic of the specific bacterium of interest using physical means (e.g., sonication, pressure, boiling or other heating means, vortexing with glass beads, etc.). Alternatively, the sample could be disrupted to make available for analysis an analyte characteristic of the specific microorganism of interest using various chemical reagents, which can include one or more components.

Methods of the present invention could be used to analyze a sample for separate molecules (e.g., molecules like protein A and Clumping Factor for analysis of Staphylococcus aureus) or two different epitopes of the same molecule (e.g., a protein). Such analytes include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin-related polysaccharides binding proteins, and the like. Protein A and clumping factors, such as fibrinogen-binding factors and clumping factors A, B, and Efb, are also particularly useful in methods of detecting the presence of Staphylococcus aureus. Other cell-wall components of interest include capsular polysaccharides and cell-wall carbohydrates (e.g., teichoic acid and lipoteichoic acid).

If desired, methods of the present invention can further include analyzing the sample for an internal cell component, which may or may not be associated with a cell membrane, as the analyte of interest. Internal cell components are particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

Thus, although whole cells are preferred for analysis using the methods of the present invention, in certain embodiments, methods of the present invention include lysing the cells in the test sample. In the methods of the present invention, lysing can include contacting the cells with a lysing agent or physically lysing the cells. Lysing can be conducted under conventional conditions, such as, for example, at a temperature of 5° C. to 42° C. (probably as high as 50° C.), preferably at a temperature of 15° C. to 25° C.

Lysing can occur upon physically lysing the cells. Physical lysing can occur upon vortexing the test sample with glass beads, sonicating, heating and boiling, or subjecting the test sample to high pressure, such as occurs upon using a French press, for example.

Lysing can also occur using a lysing agent. Suitable lysing agents include, for example, enzymes (e.g., proteases, glycosidases, nucleases). Exemplary enzymes include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. Lysostaphin is particularly useful in methods of detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), tris(2-carboxyethyl) phosphine hydrochloride (TCEP; Pierce Chemical Company, Rockford, Ill.), cysteine, n-acetyl cysteine), acids (e.g., HCl), and bases (e.g., NaOH). Such lysing agents may be more suitable for certain organisms than for others, for example, they can be more suitable for use with Gram negative bacteria than with Gram Positive bacteria.

Various combinations of such lysing agents can be used if desired.

Methods of lysing are further discussed in U.S. Patent Application Publication No. 2005/0153370. In particular, such lysing methods involve detecting one or more components of cell walls that are characteristic of a bacterium of interest, and optionally one or more internal cell components that are further characteristic of a species of interest (e.g., an antibiotic resistant microbe of interest). It is believed that the cell-wall fragments analyzed are solid pieces of cell wall. That is, it is believed that they are not solubilized upon lysing; rather, the cell wall is merely broken into pieces. Furthermore, the cell-wall component that is analyzed is still part of (i.e., in or on) the cell wall fragments. That is, they are not solublized upon lysing. Significantly, this enhances the signal of the cell-wall component relative to the same component in an unlysed cell.

One example is if S. aureus is present, the lysed cells in the test sample can be analyzed for protein A, which is characteristic for S. aureus and can be detected with a protein A specific antibody immobilized on the biosensor surface. Additionally, lysed cells, such as S. aureus bacteria, release protein markers from the internal portion of the cells (as opposed to the cell-wall portion of the cells). Such protein markers can be detected by probes, such as an antibody.

Additionally, if desired, and the sample is a mucus-containing sample, it can be further treated, either before or after lysing, with at least one reagent that can include a mucolytic agent. Treatment of mucus-containing samples with mucolytic agents can reduce the interference resulting from the presence of mucus during the analysis.

Examples of mucolytic agents include enzymes (e.g., pepsin, DNases, RNases, glucosidases, galactosidases, glycosidases), salts (e.g., chaotrophic salts), solubilizing agents (e.g., surfactants, detergents), reducing agents (e.g., beta-mercapto ethanol (BME), dithiotreotol (DTT), dithioerythritol (DTE), cysteine, tris(2-carboxyethyl) phosphine hydrochloride (TCEP; Pierce Chemical Company, Rockford, Ill.), n-acetyl cysteine), and acids (e.g., HCl). Various combinations of such mucolytic agents can be used if desired. One of skill in the art will understand that there can be overlap between lysing agents and mucolytic agents; although not all lysing agents will be mucolytic, for example.

In certain embodiments, if the sample is a mucus-containing sample, and the mucolytic agent is a reducing agent, the reducing agent may be acidified (e.g., having a pH of less than 3). Reducing agents can be acidified using a variety of acids, such as inorganic acids (e.g., HCl) or organic acids (e.g., lactic acid, citric acid). Alternatively, if used in sufficiently high concentrations, the pH of the reducing agent does not need to be adjusted with an acid. Also, alternatively, an acid alone (e.g., HCl) can be used as the mucolytic agent.

In certain embodiments, the mucosal sample and an enzymatic-lysing agent are incubated for a time sufficient to allow lysis of cells and release of at least some antigenic components of the cells; subsequently, the sample and enzymatic-lysing agent are combined with a mucolytic agent that is distinct from the enzymatic-lysing agent.

Typically, but optionally, after adding a reducing agent, the sample preparation involves inactivating the reducing agent in the composition. The terms “inactivate” or “inactivating” or “inactivation” refer to stopping the activity of a reagent or stopping a reaction, for example, which can occur by a wide variety of mechanisms, including, for example, blocking, diluting, inhibiting, denaturing, competing, etc.

Inactivating can be done, for example, by providing a competitive substrate (for example, bovine serum albumen for n-acetyl cysteine). Other examples of reagents that inactivate the reducing agent include a diluent including a neutralizing buffer. Representative ingredients for neutralizing buffers can include, for example, buffering agent(s) (e.g., phosphate), salt(s) (e.g., NaCl), protein stabilizer(s) (e.g., BSA, casein, serum) polymer(s), saccharides, and/or detergent(s) or surfactant(s) (e.g., one or more of the following agents listed by tradenames and commonly available sources: NINATE 411 (amine alkylbenzene sulfonate, available from Stepan Co., Northfield, Ill.), ZONYL FSN 100 (Telomer B monoether with polyethylene glycol, available from E.I. DuPont de Nemours Co.), Aerosol OT 100% (sodium dioctylsulfosuccinate, available from American Cyanamide Co.), GEROPON T-77 (sodium N-oleyl-N-methyltaurate, available from Rhodia Novacare), BIO-TERGE AS-40 (sodium olefin (C₁₄-C₁₆)sulfonate, available from Stepan Co.), STANDAPOL ES-1 (sodium polyoxyethylene(1) laurylsulfate, available from Cognis Corp., Ambler, Pa.), TETRONIC 1307 (ethylenediamine alkoxylate block copolymer, available from BASF Corp.), SURFYNOL 465, 485, and 104 PG-50 (all available from Air Products and Chemicals, Inc.), IGEPAL CA210 (octylphenol ethoxylate, available from Stepan Co.), TRITON X-45, X-100, and X-305 (octylphenoxypolyethoxy ethanols, all available from The Dow Chemical Co.), SILWET L-7600 (polydimethylsiloxane methylethoxylate, available from Momentive Performance Materials, Inc., Wilton, Conn.), RHODASURF ON-870 (polyethoxylated(2) oleyl alcohol, available from Rhodia Novacare), CREMOPHOR EL (polyethyoxylated castor oil, available from BASF Corp.), TWEEN 20 and TWEEN 80 (polyoxyethylene sorbitan monolaurate and monooleate, both available from Sigma-Aldrich Corp.), BRIJ 35 (polyoxyethylene(23) dodecyl ether, available from Sigma-Aldrich Corp.), CHEMAL LA-9 (polyoxyethylene(9) lauryl alcohol, available from PCC Chemax, Piedmont, S.C.), PLURONIC L64 (poly(oxyethylene-co-oxypropylene) block copolymer, available from BASF Corp.), SURFACTANT 10G (p-nonylphenoxypoly(glycidol), available from Arch Chemicals Inc., Norwalk, Conn.), SPAN 60 (sorbitan monostearate, available from Sigma-Aldrich Corp.), CREMOPHOR EL (a polyethoxylated castor oil, available from Sigma-Aldrich Corp.)). If desired, the neutralizing buffer can also be used to adjust the pH of the sample.

In addition to, or alternative to, a reducing agent, the sample preparation of a mucus-containing sample can include the use of one or more surfactants or detergents (e.g., subsequently to or concurrently with, the combining of the sample and the enzymatic lysing agent with the mucolytic agent). Suitable surfactants can be nonionic, anionic, cationic, or zwitterionic. Suitable examples include sodium dodecyl sulfate (SDS) and sodium lauryl sulfate (SLS). Various combinations of surfactants can be used, if desired.

Optionally, the sample preparation method can include subsequently inactivating the surfactant. This can be done, for example, by providing a competitive substrate. Other examples of inactivating the surfactant include using reagent neutralizing buffers, such as a buffer that is sufficient to adjust the pH of the mucolytic test sample and surfactant to a pH of at least 5. Preferably, the buffer is sufficient to adjust the pH to no greater than 8.

Furthermore, if one or more of the sample preparation reagents is acidic, the subsequent composition including the analyte of interest is preferably neutralized to a pH of 7 to 7.5 or near 7.2. This can be done, for example, by providing a buffer and/or a diluent.

Other sample types of particular interest include wound exudate, urine, and cultured blood. Wound exudate samples can be typically acquired using a swab or a similarly designed sample acquisition device to contact a wound that has been cleansed using a saline wash. The swab sample can be eluted in an extraction solution. Such extraction (i.e., elution) solutions typically include water and can optionally include a buffer and at least one surfactant. An example of an elution buffer includes, for example, phosphate buffered saline (PBS) with TWEEN 20 or with PLURONIC L-64. Other extraction solutions function to maintain specimen stability during transport from sample collection site to sample analysis sites. Examples of these types of extraction solutions include Amies' and Stuart's transport media.

The eluted exudate test sample may be filtered prior to testing in order to remove cells and other non-bacterial components (i.e. red and white blood cells, skin cells, macroscopic debris) with sizes greater than 1 μm. The sample may be ready at this point for the assay as described herein. Other means of preparing the eluted wound exudate test sample may include adding flocculating agents to promote the precipitation of interfering proteins, while maintaining the bacteria in suspension. Another sample treatment possibility includes the use of differential lysing agents that will lyse eukaryotic cells without affecting bacterial cells. Lysing with such an agent may allow filtration with membranes smaller than 1 μm in pore size to capture and isolate the bacterial cells while flushing to waste the lysed components. The bacterial cells captured on the filter could then be eluted off that filter using an elution buffer similar to the ones described for elution of the original sample from a swab.

Physical methods may also be useful in preparing a wound exudate sample. For example, centrifugation may be used to separate interfering sample components greater in size than microbes, while maintaining the target bacteria in the supernatant. These sample treatment methods are know to those skilled in the art.

Urine samples could be treated in a slightly different manner. First, the sample may be collected using a fluid handling system rather than a swab. As such it would not necessarily require elution as described for a swab sample. However, the subsequent sample treatments including: filtration, flocculation, differential lysing, and centrifugation, as described above would be useful in coarsely separating interfering sample components from the bacteria of interest.

Cultured blood samples could be treated in a manner analogous to urine samples. For example, centrifugation is a common method used to separate red and white blood cells from plasma which is the component of interest in detection of bacterial content.

### Methods of Detection

Methods for analysis of one or more analytes according to the present invention include direct and indirect methods. Preferred methods involve indirect detection.

In one embodiment, use of the abovementioned colorimetric sensors provide direct absorption measurements or allow for visual observation with the naked eye to detect color change in the colorimetric sensor. In some cases, the probe can form a complex with the analyte which can interact directly with the sensor, yielding a direct assay where the colorimetric response is directly proportional to the concentration of analyte.

In an alternative embodiment, the present invention provides a method for indirect detection of an analyte by selection of a probe with an affinity to bind with both the receptor incorporated into the polydiacetylene assemblies and the analyte. The probe selected will demonstrate a competitive affinity with the analyte. When the analyte of interest is present, the probe will bind to the analyte rather than the receptor on the polydiacetylene backbone, resulting in a color change inversely proportional to the analyte concentration. If the analyte is absent, the probe will bind to the receptor incorporated on the polydiacetylene backbone. The probe can contact the sensor after the analyte contacts the sensor, or can be mixed with the analyte prior to the mixture contacting the sensor.

In one embodiment of an indirect detection assay, the probe and the target analyte are allowed to interact in a buffer solution, which is subsequently placed in contact with the sensor. The concentration of the probe free in the buffer is dependent on the amount of analyte target present: the higher the analyte concentration, the lower the remaining concentration of probe. Since the colorimetric response of the sensor is proportional to the amount of free probe available, the colorimetric response is inversely proportional to the analyte concentration.

In a particularly preferred embodiment of an indirect assay, a sensor component includes polydiacetylene liposomes that are configured to bind with a polymyxin B sulfate probe or other reagent to detect Gram negative or Gram positive bacteria. The polymyxin B sulfate probe is mixed with the test sample under mild agitation to bind to the bacteria. The polydiacetylene liposomes are used to detect the unbound polymyxin to indirectly detect the bacteria load of the test sample. The polydiacetylene sensor component undergoes a color change upon binding between the unbound polymyxin and the polydiacetylene liposomes where the color change is indirectly proportional to the concentration of bacteria in the test sample.

In one embodiment, the method of the invention comprises providing a test sample comprising the analyte in a buffer composition, providing a probe in a buffer composition, combining the test sample and the probe wherein the probe shows a greater binding affinity for the analyte than the receptor, and detecting the change with a biosensor.

In some assays the probe could be generated in-situ by fragmenting or otherwise lysing the analyte target. The probe could also be considered a protein or protein fragment externally present on the cell wall of an organism that is available for interaction directly with the sensor. Interaction between the probe and the analyte can operate to the exclusion of interaction with the liposome. Alternatively, the probe may interact with the analyte to form a complex with the resulting complex interacting with the liposome. The probe can be contacted with the sensor in solution or coated on a substrate.

Thus, the test sample and probe may be combined in a variety of suitable manners. In one aspect, the probe is provided to the sensor and the test sample is provided to the colorimetric sensor as separate portions, yet in any order. For example, the surface may be coated with a polymixin-containing solution and optionally dried. In another aspect, the test sample and probe are combined as a mixture and the mixture is provided to the colorimetric sensor. In a preferred embodiment, the probe interacts with the test sample containing the analyte before contacting the colorimetric sensor.

Using the indirect method of detection, high sensitivity that provides low levels of detection are possible based on the concentration of probe used. For detection strategy, probe concentrations can be chosen to correspond to desired concentration levels of detection. The method of indirect detection using the probe allows design of the system around the type and concentration of the probe for desired sensitivity in a given application. This allows the transducer to be universal to multiple analytes of interest. For example, a single transducer (polydiacetylene/receptor combination) could serve to detect multiple analytes by varying the probe in contact with the transducer in accordance with the probe's affinity for the analyte.

In certain embodiments, the colorimetric sensor can be provided in a solution or suspension in a simple vial system, wherein an analyte can be added directly to a vial containing a solution with the transducer specific to the analyte of interest. Alternatively, the system could include multiple vials in a kit, with each vial containing a transducer comprising polydiacetylene assemblies with incorporated receptors particular to different analytes.

For those applications in which the analyte cannot be added directly to the polydiacetylene transducer, a two-part vial system could be used. One compartment of the vial could contain reagents for sample preparation of the analyte physically separated from the second compartment containing the transducer formed from the polydiacetylene assemblies. Once sample preparation is complete, the physical barrier separating the compartments would be removed to allow the analyte to mix with the transducer for detection.

Alternatively, a kit could also contain a vial for reagant storage and mixing of the analyte before contacting the colorimetric sensor coated on a two-dimensional substrate. In one embodiment, the kit could comprise a vial for reagent storage and analyte preparation, with a cap system containing the transducer of the present invention coated on a substrate.

A solution or suspension of a colorimetric sensor can then be coated on a solid substrate by spotting the substrate and allowing the liquid carrier (e.g., water) to evaporate. Suitable substrates can include highly flat substrates, such as evaporated gold on atomically flat silicon (111) wafers, atomically flat silicon (111) wafers, or float glass, which are bare and modified with self-assembling monolayers (SAMs) to alter their surface energy in a systematic fashion; or substrates with a highly textured topography that include paper substrates, polymeric ink receptive coatings, structured polymeric films, microporous films, and membrane materials.

Alternatively, a solution or suspension of a colorimetric sensor can be extruded through a membrane of appropriate pore size, entrapping the polydiacetylene assemblies and resulting in a coated membrane, which is subsequently allowed to dry. Appropriate membranes are generally those with pore size of 200 nm or less, comprising materials like polycarbonate, nylon, PTFE, polyethylene (others can be listed).

These substrates can be either coated with a polymerized suspension of the diacetylene assemblies, or the suspension can be coated in the unpolymerized form and subsequently polymerized in the coated state. The coating weight of the sensor typically affects the sensitivity of the sensor. Ideally, the coating weight should be designed to bind with the analyte and undergo the detectable change in a reasonable time period. The coating weight should also preferably be uniform across the substrate to uniformly expose, for example, the test sample to the sensor component.

Various forms of the colorimetric sensor can be used, including, for example, tape or label form. See, e.g., U.S. Patent Application Publication No. 2004/132217.

In certain embodiments, the colorimetric sensors of the present invention could be paired with other known diagnostic methods to provide a multi-prong determination of the presence of bacteria or other analytes.

### EXAMPLES

The present invention should not be considered limited to the particular examples described below, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. All parts, percentages, ratios, etc. in the examples and the rest of the specification are by mole unless indicated otherwise. All solvents and reagents without a named supplier were purchased from Aldrich Chemical; Milwaukee, Wis. Water was purified by the use of a U-V Milli-Q water purifier with a resistivity of 18.2 Mohms/cm (Millipore, Bedford, Mass.).

The colorimetric response from the polydiacetylene indicator is characterized by measuring hue angle)(h°). The values of h° range from 0° to 360°, which essentially measures the RGB (red, green, blue) value of a given color. Pure red corresponds to an h° value of 0°, pure green corresponds to an h° value of 120°, and pure blue corresponds to an h° value of 240°. The color circle is continuous, therefore there is no discontinuity going from 360° to 0° (both values correspond to pure red). On average, the dynamic range of a preferred polydiacetylene indicator covers the interval of hue angles from approximately 260° (blue phase) to approximately 360° (red phase). The h° values were determined by direct measurements of the color using a commercial spectrophotometer (Avantes AvaSpec-2048-SPU2-SD256 available from Wilkens-anderson Co., Chicago, Ill.).

Table of Abbreviations

| Abbreviation or | |
|---|---|
| Trade Name | Description |
| ATCC | American Type Culture Collection |
| DMPC | 1,2-dimeristoyl-sn-glycero-3- |
| | phosphocholine (DMPC, formula weight |
| | (F.W.) 678, available from Sigma-Aldrich, |
| | St. Louis, MO |
| HEPES | N-2-Hydroxyethylpiperazine-N′-2- |
| | ethanesulfonic acid available from Sigma- |
| | Aldrich, St. Louis, MO |
| PBS buffer | A phosphate buffer saline (PBS) solution |
| | prepared by diluting ten-fold a 10x PBS |
| | liquid concentrate available from EMD |
| | Biosciences, San Diego, CA |
| PBS L64 buffer | prepared by taking the PBS buffer solution |
| | and adding 0.2% (w/v) of PLURONIC L64 |
| PLURONIC L64 | Trade designation for surfactant available |
| | from BASF Corporation, Mount Olive, NJ |

### Preparative Example 1

### Preparation of Antibody Functionalized Magnetic Beads

Murine anti-Protein A monoclonal antibody, MAb-107, is described in U.S. patent application Ser. No. 11/562,747, filed on Nov. 22, 2006, and PCT Application No. US2007/084,739, both entitled “ANTIBODY WITH PROTEIN A SELECTIVITY.”

Antibodies for B. anthracis (catalog number J-260800-01 lots 0016N2B-8 and 00116OFQ-rabbit anti-B. anthracis) were obtained Edgewood Chemical and Biological Center, Edgewood, Md.

All antibody preparations were biotinylated with EZ-Link NHS-PEO4-Biotin (Product Number 21330) from Pierce according to the manufacturer's directions. Streptavidin-coated magnetic particles (1 μm Dynal T1) were obtained from Invitrogen, Inc. (Carlsbad, Calif.). All reactions and washes were performed in PBS L-64 buffer (phosphate buffered saline with 0.2% w/v PLURONIC L64) unless stated otherwise. Wash steps included three successive washes unless stated otherwise. The washing process consisted of placing a magnet adjacent to the tube to draw the particles to the side of the tube proximal to the magnet, removing the liquid from the tube with the adjacent magnet, and adding an equal volume of fresh buffer to replace the liquid that was removed. The magnet was removed to allow resuspension and mixing the particles.

Streptavidin-coated magnetic particles, at a concentration of 2.5 milligram per milliliter (mg/mL) were mixed with biotinylated antibody preparations in 500 microliter (μL) PBS L-64 buffer. The mass ratio of the antibody to the particles for conjugation was 40 μg antibody/mg of particles. The resulting mixture was incubated at 37° C. for 1 hour (hr). Subsequently, the particles were washed in PBS L-64 buffer to remove unbound antibody. After the final wash the particles were resuspended to a particle concentration of 2.5 mg/mL.

### Preparative Example 2

### Preparation of S. aureus Bacterial Suspension

S. aureus bacteria were obtained from The American Type Culture Collection (Rockville, Md.), under the trade designation ATCC 25923. The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS L64 buffer and washed by centrifugation for 3 additional cycles with this solution.

### Preparative Example 3

### Preparation of S. epidermidis Bacterial Suspension

S. epidermidis bacteria were obtained from The American Type Culture Collection (Rockville, Md.), under the trade designation ATCC 12228. The bacteria were grown in overnight (17-22 hours at 37° C.) broth cultures prepared by inoculating 5-10 milliliters of prepared, sterile Tryptic Soy Broth (Hardy Diagnostics, Santa Maria, Calif.) with the bacteria. Cultures were washed by centrifugation (8,000-10,000 rpm for 15 minutes in an Eppendorf model number 5804R centrifuge (Brinkman Instruments, Westbury, N.Y.) and resuspended into PBS L64 buffer and washed by centrifugation for 3 additional cycles with this solution.

### Preparative Example 4

### Preparation of B. thuringiesis Spore Suspension

B. thuringiensis organisms were obtained from The American Type Culture Collection (Rockville, Md.), under the trade designation ATCC 10792. The organisms were first cultured by streaking on a nutrient agar plate and incubating overnight at 37° C. To generate the spores, 10 mL of Shaeffers' Sporulation Medium was inoculated with material from the edge of about 3 to 5 isolated colonies. Vortexing was used to suspend the cells completely. The suspension was incubated at 37° C. under agitation for 18 hours. Organisms were harvested when most of the cells contained spores and before lysis of a significant number of cells had occurred. The spores were collected by centrifugation (Eppendorf model number 5804R centrifuge available from Brinkman Instruments, Westbury, N.Y.) for 30 minutes at 15,000×g and 4° C., and suspended in 35 mL cold (4° C.) distilled H₂O, Spores were then centrifuged for 10 minutes at 6,000×g and 4° C., and washed four times in cold (4° C.) distilled H₂O. Following cold distilled water washes, spores were centrifuged once more for 10 minutes at 6,000×g and 4° C., and suspended in 10 mL cold (4° C.) 1M KHPO₄ buffer (pH 7.1). Spores were then transferred to 12 mL of cold (4° C.) polyethylene glycol (PEG) in 1M KHPO₄ buffer (pH 7.1) to remove vegetative cells and debris by centrifugation for 2 minutes at 1,500×g and 4° C. Spores within the upper PEG layer were removed and additional extractions with 5 mL PEG were performed for a total of 7 extractions. PEG purified spores were collected by centrifugation for 15 minutes at 12,000×g and 4° C., and washed 7 times with 35 mL cold (4° C.) distilled water. Purified spore preparations were stored at 4° C. in HPLC grade H₂O.

### Preparative Example 5

### Preparation of the Hepes Buffer

A N-2-Hydroxyethylpiperazine-N′-2-ethanesulfonic acid (HEPES) buffer was prepared by dissolving 1.30 grams (g) of the HEPES sodium salt (F.W. (260.29) available from Aldrich Chemical; Milwaukee, Wis.) in 1 L of water. This yields a buffer salt concentration of 5 mM. The buffer solution was then titrated to pH=7.2 by dropwise adding either hydrochloric acid or acetic acid (both available from Aldrich Chemical; Milwaukee, Wis.).

### Preparative Example 6

### Preparation of the Phosphate Buffer Saline with PLURONIC L64 Buffer (PBS-L64 Buffer)

A phosphate buffer saline (PBS) solution was prepared by diluting ten-fold a 10×PBS liquid concentrate (available commercially from EMD Biosciences, San Diego, Calif.). This results in a PBS buffer solution with the following salt composition: 10 mM Sodium Phosphate, 137 mM Sodium Chloride, 2.7 mM Potassium Chloride. The PBS buffer solution has a pH of 7.5 at 25° C. To prepare the Phospate Buffer Saline with PLURONIC L64 solution (PBS-L64 buffer solution), 0.2% (w/v) of the PLURONIC L64 surfactant (available from BASF Corporation, Mount Olive, N.J.) was added to the PBS buffer solution. The PBS-L64 buffer solution has a pH of 7.5 at 25° C.

### Preparative Example 7

### Preparation of Polymyxin B Sulfate Solution

A stock solution of Polymyxin B Sulfate (PmB, formula weight (F.W.) 1385, available from Sigma-Aldrich, St. Louis, Mo.) was prepared by adding 7.21 mg of PmB to 10 mL of HEPES buffer (as prepared in Preparative Example 5) under stirring until complete dissolution of the peptide was achieved. This yields a final Polymyxin B Sulfate solution concentration of 520 nmoles/mL.

### Preparative Example 8

### Preparation of Polydiacetylene Liposome Suspension

Diacetylene, HO(O)C(CH₂)₂C(O)O(CH₂)₄C≡C—C≡C(CH₂)₄O(O)C(CH₂)₁₂CH₃, was prepared as in Example 6 of U.S. Patent Application Publication No. 2004/0132217. The basic procedure involved reacting 5,7-dodecadiyn-1,12-diol (HO(CH₂)₄C≡C—C≡C(CH₂)₄OH) with myristol chloride and subsequent reaction of that product with succinic anhydride to yield the diacetylene, HO(O)C(CH₂)₂C(O)O(CH₂)₄C≡C—C≡C(CH₂)₄O(O)C(CH₂)₁₂CH₃, as a white solid.

A (6:4) mixture of the diacetylene compound: HO(O)C(CH₂)₂C(O)O(CH₂)₄C≡C—C≡(CH₂)₄O(O)C(CH₂)₁₂CH₃ (succinic acid mono-(12-tetradecanoyloxy-dodeca-5,7-diynyl) ester), and the zwitterionic phospholipid 1,2-dimeristoyl-sn-glycero-3-phosphocholine (DMPC, formula weight (F.W.) 678, available from Sigma-Aldrich, St. Louis, Mo.) was weighed into a glass vial and suspended in water (pH 5.8) to produce a 1 mM solution. This solution was then probe sonicated using a Misonix XL202 probe sonicator (available commercially from Misonix Inc., Farmington, N.Y.) for 2 minutes, and placed in a 4° C. refrigerator for about 20 hours. This process results in the formation of a stable liposome suspension.

### Preparative Example 9

### Polymerization of the Diacetylene Liposome Suspension

The suspension prepared in Preparative Example 8 was polymerized by first diluting 1:10 in water, and then UV exposing the diluted sample using a Fusion UV Systems (Gaithersburg, Md.) high power (250 mJoule) UV station (3 passes at 50 ft/min (0.254 meters/second) under 254-nm wavelength). Alternatively, the suspension prepared in Preparative Example 6 can be polymerized by diluting 1:10 in water and irradiating the diluted sample beneath a 254 nm UV lamp (commercially available from VWR Scientific Products; West Chester, Pa.) at a distance of 3 cm for 35 minutes while stirring. Both methods result in the observation of a blue color (blue phase), with a hue angle typically in the range of 260° to 270°. Polymerizations where typically carried out using 10 mL volumes of the diluted liposome suspension.

### Preparative Example 10

### Preparation of the Fluidic System

The fluidic system used in our assay is essentially a flow-through system. The system consists of a blank 96-well 3M Empore Filter Plate (No. 6060, Filter PPT small volume 96-well extraction plate available from 3M Filtration Products, St. Paul, Minn.), where each well is loaded with a 1-centimeter (cm) diameter disk of HT Tuffryn 450 membrane (hydrophilic polysulfone 450 nm membrane available from Pall Corporation, Ann Arbor, Mich.) punched out from a larger sheet of the membrane and masked using a vinyl tape (Scotch Super 33 Plus Vinyl Electrical Tape available from 3M Company, St. Paul, Minn.) so as to precisely define a filtration area of 8 mm². The masked membrane disk is held against the bottom of each well in the 96-well plate by using a polypropylene ferrule (available from 3M Filtration Products, St. Paul, Minn.). The plate as prepared is used with a vacuum manifold (available from 3M Filtration Products, St. Paul, Minn.), adjusting the vacuum to yield a flow rate between 100 and 250 μL/min. The liposome suspension resulting from the completed assay is filtered using this fluidic system forming a coating on the membrane disk at the bottom of the well. The hue angle of the liposome coating is measured directly by using a commercial spectrophotometer (Avantes AvaSpec-2048-SPU2-SD256 available from Wilkens-anderson Co., Chicago, Ill.) outfitted with a fiber optic probe that fits inside the wells of the 96-well plate.

### Preparative Example 11

### Preparation of Coated Samples of the Diacetylene Liposome Suspension

The suspension prepared in Preparative Example 8 was coated onto a porous polycarbonate membrane with 200 (nm) diameter pores (available from Avestin, Inc. Ottawa, Canada) using a Biodot coater (available from Biodot Corporation, Irvine, Calif.) at a coating weight of 100 μL/cm². The coated membrane was placed coated side up on a glass slide and placed in a refrigerator at 5° C. for at least 3 hours. The sample was then dried in a dessiccator containing CaSO₄ for 30 minutes and exposed to 254 nanometer (nm) UV light (commercially available from VWR Scientific Products; West Chester, Pa.) for 30-90 seconds to polymerize the coated diacetylene liposomes. Round samples 1 cm in diameter were punched out from the coated, polymerized membrane and laminated to the bottom of a polycarbonate 24-well plate (available from VWR Scientific Products; West Chester, Pa.) using double-sided tape (available from 3M Stationary Products Division, St. Paul, Minn.).

### Preparative Example 12

### Preparation of Chlorhexidine diAcetate Solution

A stock solution of Chlorhexidine diAcetate (CHdiA, formula weight (F.W.) 625.55, available from Sigma-Aldrich, St. Louis, Mo.) was prepared by adding 1.564 mg of CHdiA to 100 mL of HEPES buffer (as prepared in Preparative Example 5) under stirring until complete dissolution of the solid was achieved. This yields a final CHdiA solution concentration of 25 nmoles/mL.

### Examples 1-15

### Detection of S. aureus

The assay to detect S. aureus was conducted as follows:
(1) Thirty two (32) μL of the magnetic bead suspension functionalized with MAb 107 antibody (as prepared in Preparative Example 1) were added to a polypropylene microcentrifuge tube (available from VWR Scientific, West Chester, Pa.). To the same microcentrifuge tube were added 468 μL of the S. aureus bacterial suspension in PBS-L64 buffer (as prepared in Preparative Examples 2 and 6) at a given concentration (reported in Table 1). The mixture was incubated, under rocking agitation using a Barnstead LabQuake shaker (available from Barnstead International, Dubuque, Iowa), for 15 minutes at room temperature.
(2) The beads were then separated and concentrated by placing the microcentrifuge tube in a Dynal magnetic fixture (available from Invitrogen, Inc. Carlsbad, Calif.) for at least 5 minutes. The supernatant was discarded by micropipetting without disrupting the agglomerated beads.
(3) The beads were then washed by adding 0.5 mL HEPES buffer (as described in Preparative Example 5) to the tube and agitating using a rocking motion for 5 minutes. The beads were then again separated and concentrated by placing the microcentrifuge tube in a Dynal magnetic fixture for at least 5 minutes. The wash solution was discarded by micropipetting without disrupting the agglomerated beads. This wash step was repeated a second time.
(4) A given volume (reported in Table 1) of the Polymyxin B stock solution (as prepared in Preparative Example 7) was added to the washed magnetic beads. The microcentrifuge tube was then vortexed for 10 seconds and allowed to stand for 5 minutes. Next, 0.5 mL of HEPES buffer was added to the tube and the solution was agitated for an additional 5 minutes.
(5) The beads were then again separated and concentrated by placing the microcentrifuge tube in a Dynal magnetic fixture for at least 5 minutes. The supernatant was then micropipetted to a new microcentrifuge tube, and to that was added a given volume of the PDA liposome solution (as prepared in Preparative Examples 8 and 9) as reported in Table 1. The tube was agitated gently for 1 minute.
(6) The solution was pipetted into one of the wells of the 3M Empore 96-well plate (as prepared in Preparative Example 10) and filtered under vacuum to capture and concentrate the PDA liposomes. Once the entire volume of the sample solution was filtered, the AVENTIS instrument (as described in Preparative Example 10) was used to measure the hue angle. The average hue angle)(h°) from three replicates is reported in Table 1. Also reported in Table 1 is the Colorimetric Response which is given by the following equation:

$$CRh = \frac{HueAngel ( NegativeControl ) - HueAngle ( Sample )}{HueAngel ( NegativeControl ) - HueAngle ( PositiveControl )} ,$$

where: Example 1 serves as the positive control (Hue Angle(Positive Control)) for all other experiments, and Examples 2-6 are the negative controls (Hue Angle(Negative Control)) for the corresponding Examples 7-15 (Hue Angle(Sample)). Large colorimetric responses indicate positive detection of the bacteria. 1σ standard deviations on the reported values of the colorimetric response are ±10%. These examples demonstrate the ability to detect a target organism over a range of concentrations, for different combinations of reagents (PmB) and PDA liposomes.

TABLE 1

| | | | PDA | | |
|---|---|---|---|---|---|
| | S. aureus | PmB | Liposome | Hue | Colorimetric |
| | Concentration | Volume | Volume | Angle | Response |
| Example | (cfu/mL) | (μL) | (μL) | (°) | (%) |
| 1 | 0 | 0 | 15 | 256.8 | — |
| 2 | 0 | 33 | 15 | 294.6 | — |
| 3 | 0 | 33 | 10 | 320.3 | — |
| 4 | 0 | 44 | 12.5 | 342.5 | — |
| 5 | 0 | 55 | 15 | 331.0 | — |
| 6 | 0 | 55 | 10 | 357.0 | — |
| 7 | 10000 | 33 | 15 | 261.7 | 87 |
| 8 | 10000 | 33 | 10 | 278.0 | 67 |
| 9 | 10000 | 55 | 15 | 328.8 | 3 |
| 10 | 10000 | 55 | 10 | 351.0 | 6 |
| 11 | 1000000 | 33 | 15 | 256.7 | 100 |
| 12 | 1000000 | 33 | 10 | 256.3 | 101 |
| 13 | 1000000 | 55 | 15 | 292.0 | 53 |
| 14 | 1000000 | 55 | 10 | 307.2 | 50 |
| 15 | 100000 | 44 | 12.5 | 318.4 | 28 |

### Examples 16-20

### Detection of S. aureus in the Presence of S. Epidermidis

The assay to detect S. aureus in the presence of S. epidermidis was conducted as described for Examples 1-15 with the inclusion of a sample were S. epidermidis (as prepared in Preparative Example 3) was mixed into the solution containing S. aureus in order to demonstrate the detection of a target analyte (S. aureus) in the presence of a significant concentration of an interfering organism (S. epidermidis). The average hue angle)(h°) from three replicates and the corresponding colorimetric response are reported in Table 2. 1σ standard deviations on the reported values of the colorimetric response are ±15%.

TABLE 2

| | S. aureus | S. epidermidis | PmB | PDA Liposome | Hue | Colorimetric |
|---|---|---|---|---|---|---|
| | Conc. | Conc. | Volume | Volume | Angle | Response |
| Example | (cfu/mL) | (cfu/mL) | (μL) | (μL) | (°) | (%) |
| 16 | 0 | 0 | 0 | 15 | 255.3 | — |
| 17 | 0 | 0 | 50 | 15 | 358.2 | — |
| 18 | 0 | 1000000 | 50 | 15 | 362.6 | −4.3 |
| 19 | 1000000 | 1000000 | 50 | 15 | 240.3 | 114.6 |
| 20 | 1000000 | 0 | 50 | 15 | 252.1 | 103.1 |

### Examples 16-20

### Detection of B. thuringiensis

The assay to detect B. thuringiensis was conducted as described for Examples 1-15 using J-260800-01 antibody functionalized magnetic beads (as prepared in Preparative Example 1) and B. thuringiensis (as prepared in Preparative Example 4) as the target organism. These examples demonstrate the ability to detect a different target organism by substitution of the appropriate sample preparation system. The average hue angle)(h°) from three replicates and the corresponding colorimetric response are reported in Table 3. 1σ standard deviations on the reported values of the colorimetric response are ±10%.

TABLE 3

| | B. | | PDA | | |
|---|---|---|---|---|---|
| | thuringiensis | PmB | Liposome | Hue | Colorimetric |
| | Concentration | Volume | Volume | Angle | Response |
| Example | (cfu/mL) | (μL) | (μL) | (°) | (%) |
| 21 | 0 | 0 | 15 | 258.6 | — |
| 22 | 0 | 24 | 15 | 277.2 | — |
| 23 | 1000000 | 24 | 15 | 264.8 | 33.3 |
| 24 | 0 | 36 | 15 | 282.9 | — |
| 25 | 1000000 | 36 | 15 | 269.0 | 57.2 |
| 26 | 0 | 48 | 15 | 327.6 | — |
| 27 | 1000000 | 48 | 15 | 306.2 | 31.0 |

### Examples 28-31

### Detection of S. aureus Using CHG Instead of PmB and Coated PDA Indicators

The assay to detect S. aureus was conducted as follows:
(1) Thirty two (32) μL of the magnetic bead suspension functionalized with MAb 107 antibody (as prepared in Preparative Example 1) were added to a polypropylene microcentrifuge tube (available from VWR Scientific, West Chester, Pa.). To the same microcentrifuge tube were added 468 μL of the S. aureus bacterial suspension in PBS-L64 buffer (as prepared in Preparative Examples 2 and 6) at a given concentration (reported in Table 1). The mixture was incubated, under rocking agitation using a Barnstead LabQuake shaker (available from Barnstead International, Dubuque, Iowa), for 15 minutes at room temperature.
(2) The beads were then separated and concentrated by placing the microcentrifuge tube in a Dynal magnetic fixture (available from Invitrogen, Inc., Carlsbad, Calif.) for at least 5 minutes. The supernatant was discarded by micropipetting without disrupting the agglomerated beads.
(3) The beads were then washed by adding 0.5 mL HEPES buffer (as described in Preparative Example 5) to the tube and agitating using a rocking motion for 5 minutes. The beads were then again separated and concentrated by placing the microcentrifuge tube in a Dynal magnetic fixture for at least 5 minutes. The wash solution was discarded by micropipetting without disrupting the agglomerated beads. This wash step was repeated a second time.
(4) One (1) mL of the CHG stock solution (as prepared in Preparative Example 12) was added to the washed magnetic beads. The microcentrifuge tube was then vortexed for 10 seconds and allowed to stand for 5 minutes. The beads were then again separated and concentrated by placing the microcentrifuge tube in a Dynal magnetic fixture for at least 5 minutes. The supernatant was then micropipetted into one of the wells of the 24-well plate (as prepared in Preparative Example 11) containing a coated PDA indicator. The 24-well plate was agitated on an Eberbach Model 6000 shaker (Eberbach Corp., Ann Arbor, Mich.). A picture was taken at 60 minutes using a digital camera. The picture was analyzed using software from Adobe Systems Incorporated (trade designation ADOBE PHOTOSHOP version 5.0, San Jose, Calif.) to extract the hue angle of the PDA indicator. The average hue angle)(h°) from three replicates and the corresponding colorimetric response reported in Table 4. 1σ standard deviations on the reported values of the colorimetric response are ±10%. These examples demonstrate the ability to detect a target organism using a different reagent (CHG instead of PmB) and coated indicator (solid phase versus solution phase detection).

TABLE 4

| | S. aureus | CHG | Hue | Colorimetric |
|---|---|---|---|---|
| | Conc. | Volume | Angle | Response |
| Example | (cfu/mL) | (mL) | (°) | (%) |
| 28 | 0 | 0 | 265.3 | — |
| 29 | 0 | 1 | 361.5 | — |
| 30 | 1000 | 1 | 348.7 | 13.3 |
| 31 | 1000000 | 1 | 283.1 | 81.5 |

### Examples 32-34

### Detection of S. aureus in a Disposable Detection Device for Point of Care Applications

FIG. 3 illustrates a detection device 450 having a sensor layer or portion 130 and flow-through membrane 460 where a body of the device is formed of a multiple layer construction. As shown, the multiple layer construction includes a face or first outer layer 454, a backing or second outer layer 456 and one or more intermediate layers. In the embodiment shown, the sensor component 100 is supported proximate to an opening 457 through intermediate layer 458. Sensor layer or portion 130 is disposed on membrane 460, which is coupled to the intermediate layer 458 proximate to opening 457. The intermediate layer 458 is water impermeable. The multiple layered structure also includes a spacer layer 462 disposed between the face layer 454 and intermediate layer 458. The spacer layer 462 is patterned to form inlet 464 and the first flow path portion. An absorbent layer 466 is disposed between the intermediate layer 458 and the backing layer 456 proximate to the opening 457 to induce fluid flow across a sensor passageway formed through the flow-through membrane 460 in opening 457.

As described, the first flow path portion is formed of a passage orientated along a length of the multiple layered structure between the face layer 454 and the intermediate layer 458 to provide flow in a first direction. The device also includes a second flow path portion formed traverse to the first flow path portion to provide flow in a second direction generally transverse to the first direction across the flow-through membrane 460. In the illustrated embodiment, the face layer 454 is formed of a transparent or see-through film so that the sensor component 100 is visible to discern the detectable change upon reaction of the analyte with the sensor component 100. Alternatively a portion of the face layer 454 is transparent or see-through to view the sensor component 100.

In the illustrated embodiment, fluid flow is induced across the flow-through membrane 460 via the absorbent layer 466. Layer 466 can be patterned to form an absorbent area downstream of the flow-through membrane 460 to form the traverse flow path or passage. Although FIG. 3 illustrates a separate backing or outer layer 456, in alternate embodiments, the absorbent layer 466 can form the backing layer of the device, and application is not limited to the specific layers shown.

In each of the illustrated embodiments a time or period of exposure of the test sample to the sensor component 100 is limited based upon the flow rate of the test sample across the sensor component 100. Once the fluid flows past the sensor component 100 it is no longer exposed to the sensor layer or portion, thus limiting exposure of the test sample to the sensor component 100 to provide a relatively stable test result which does not vary significantly following conclusion of the test.

For these examples, the device described above was constructed using the following materials: layer 456 was a vinyl tape (Scotch Super 33 Plus Vinyl Electrical Tape available from 3M Company, St. Paul Minn.), layer 466 was a glass fiber wicking material (Sterlitech GB 140 Glass Fiber, available from Sterlitech Corporation, Kent Wash.), layer 460 is a 450 nm porosity polyethersulfone membrane (Pall Supor 450 Membrane, available from Pall Corporation, Ann Arbor Mich.), layer 458 is a 1/32-inch thick PVC backing material with a pressure sensitive adhesive on one side (Diagnostic Consulting Network Miba-010, available from Diagnostic Consulting Network, Irvine Calif.), layer 462 is a 1/16-inch thick 3M Polyethylene blown foam with a pressure sensitive adhesive on both sides (available from 3M Medical Division, 3M Company, St. Paul Minn.), and layer 454 is a 3M Polyester General Use Transparency Film (available from 3M Company, St. Paul Minn.).

To construct the detection device, each of the film layers was die cut to its proper shape and size using a rotary die. The assembly begins by placing the flow-through filter membrane 460 over the opening 457 on the adhesive side of the intermediate layer 458. Next, the absorbent layer 466 is placed over the filter membrane and positioned over the opening 457 on the adhesive side of the intermediate layer 458. This initial laminate was then placed absorbent layer 466 down on the adhesive side of the backing layer 456, applying pressure at the edges to ensure that the backing layer 456 adheres around the absorbent layer 466 to the intermediate layer 458, forming a seal. Next, the liner from one side of the spacer layer 462 was removed and the adhesive side of the spacer layer 462 was laminated to the non-adhesive side of the intermediate layer 458. Finally, the liner from the other side of the spacer layer 462 is removed, and the outer layer 454 is laminated to the adhesive layer on the spacer layer 462. A needle was used to create two vent holes located at the top of the sample chamber.

To test the detection device, assays to detect S. aureus were carried out according to the following protocol:
(1) Thirty two (32)μL of the magnetic bead suspension functionalized with MAb 107 antibody (as prepared in Preparative Example 1) were added to a polypropylene microcentrifuge tube (available from VWR Scientific, West Chester Pa.). To the same microcentrifuge tube were added 484 μL of the S. aureus bacterial suspension in PBS-L64 buffer (as prepared in Preparative Examples 2 and 6) at a given concentration (reported in Table 1). The mixture was incubated, under rocking agitation using a Barnstead LabQuake shaker (available from Barnstead International, Dubuque Iowa), for 15 minutes at room temperature.
(2) The beads were then separated and concentrated by placing the microcentrifuge tube in a Dynal magnetic fixture (available from Invitrogen, Inc. Carlsbad, Calif.) for at least 5 minutes. The supernatant was discarded by micropipetting without disrupting the agglomerated beads.
(3) The beads were then washed by adding 0.5 mL HEPES buffer (as described in Preparative Example 5) to the tube and agitating using a rocking motion for 5 minutes. The beads were then again separated and concentrated by placing the microcentrifuge tube in a Dynal magnetic fixture for at least 5 minutes. The wash solution was discarded by micropipetting without disrupting the agglomerated beads. This wash step was repeated a second time.
(4) A given volume (reported in Table 1) of the Polymyxin B stock solution (as prepared in Preparative Example 7) was added to the washed magnetic beads. The microcentrifuge tube was then vortexed for 10 seconds and allowed to stand for 5 minutes. Next, 0.5 mL of HEPES buffer was added to the tube and the solution was agitated for an additional 5 minutes.
(5) The beads were then again separated and concentrated by placing the microcentrifuge tube in a Dynal magnetic fixture for at least 5 minutes. The supernatant was then micropipetted to a new microcentrifuge tube, and to that was added a given volume of the PDA liposome solution (as prepared in Preparative Examples 8 and 9) as reported in Table 5. The tube was agitated gently for 1 minute.
(6) The solution was pipetted into one of the detection devices. At this point the solution starts to filter through the flow-through membrane by capillary action, and the PDA liposomes are collected and concentrated on that membrane. Once the entire volume of the sample solution was filtered and the sample chamber had completely drained, the Aventis™ instrument (as described in Preparative Example 10) was used to measure the hue angle. The average hue angle)(h°) from three replicates is reported in Table 5. Also reported in Table 5 is the Colorimetric Response which is given by the following equation:

$$CRh = \frac{HueAngel ( NegativeControl ) - HueAngle ( Sample )}{HueAngel ( NegativeControl ) - HueAngle ( PositiveControl )} ,$$

where: Example 32 serves as the positive control (Hue Angle(Positive Control)) for all other experiments, and Example 33 is the negative control (Hue Angle(Negative Control)) for the corresponding Example 34 (Hue Angle(Sample)). Large colorimetric responses indicate positive detection of the bacteria. 16 standard deviations on the reported values of the colorimetric response are ±10%. These examples demonstrate the ability to detect a target organism in the detection device.

TABLE 5

| | | | PDA | | |
|---|---|---|---|---|---|
| | S. aureus | PmB | Liposome | Hue | Colorimetric |
| | Concentration | Volume | Volume | Angle | Response |
| Example | (cfu/mL) | (uL) | (uL) | (°) | (%) |
| 32 | 0 | 0 | 15 | 263.2 | — |
| 33 | 0 | 33 | 15 | 301.4 | — |
| 34 | 1000000 | 33 | 15 | 267.6 | 88 |

The complete disclosures of all patents, patent applications, publications, and nucleic acid and protein database entries, including for example GenBank accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A method of analyzing a sample for a bacterium, the method comprising:
providing a sample suspected of including one or more distinct analytes characteristic of a specific bacterium;
providing one or more antibodies having antigenic specificities for one or more distinct analytes characteristic of the specific bacterium;
providing a solid support material;
providing contact between the sample, the solid support material, and the one or more antibodies under conditions effective to capture one or more analytes characteristic of a specific bacterium, if present;
providing a colorimetric sensor comprising a polymerized composition comprising a diacetylene-containing polymer and a receptor, wherein the receptor is incorporated in the polymerized composition to form a transducer that provides a color change upon binding with one or more probes and/or analytes;
optionally, removing the one or more analytes, if present, from the solid support material; and
subsequent to capture and optional removal of the one or more analytes, subjecting the one or more analytes, if present, to direct or indirect analysis by the colorimetric sensor to analyze for the presence or absence of the specific bacterium.

2. The method of claim 1, wherein providing contact between the sample, the solid support material, and the one or more antibodies comprises providing simultaneous contact between the sample, the solid support material, and the one or more antibodies.

3. The method of claim 1, wherein the one or more antibodies are attached to the solid support material forming an analyte-binding material, and the method includes providing contact between the sample and the analyte-binding material under conditions effective to capture one or more analytes characteristic of a specific bacterium, if present.

4. The method of claim 3, wherein the analyte-binding material comprises two or more antibodies having antigenic specificities for two or more distinct analytes characteristic of the specific bacterium.

5. The method of claim 4, wherein the antibodies are monoclonal, polyclonal, or combinations thereof.

6. The method of claim 4, wherein the antibodies are selected from the group consisting of MAb-76, MAb-107, affinity-purified RxClf40, affinity-purified GxClf40, MAb 12-9, fragments thereof, and combinations thereof.

7. The method of claim 3, wherein the analyte-binding material comprises particulate material comprising at least two portions, wherein one portion of particulate material has one antibody specific for one analyte disposed thereon, and a second portion has a different antibody specific for a distinct analyte disposed thereon.

8. The method of claim 3, wherein the solid support material comprises particulate material, wherein each particle of the particulate material has at least two antibodies that bind different analytes disposed thereon.

9. The method of claim 1, wherein the solid support material comprises magnetic particles.

10. The method of claim 1, wherein further comprising providing a buffer composition that mediates the interaction between the analyte(s) and the transducer.

11. The method of claim 1, wherein the one or more analytes characteristic of a specific bacterium are present on whole cells.

12. The method of claim 1, wherein the specific bacterium comprises a Gram positive bacterium.

13. The method of claim 12, wherein the specific bacterium comprises Staphylococcus aureus.

14. The method of claim 1, comprising subjecting the one or more analytes, if present, to direct analysis by the colorimetric sensor.

15. The method of claim 1, further comprising the use of one or more probes in an indirect assay.

16. The method of claim 1, further comprising:
providing one or more probes;
providing conditions effective for the probes to bind to the one or more analytes, if present, before capture, after capture, or after optional removal from the solid support material; and
providing contact between the unbound probes and the colorimetric sensor to analyze for the presence or absence of the specific bacterium.

17. The method of claim 16, wherein the one or more probes comprise a polymyxin.

18. The method of claim 1, wherein the sample is a mucus-containing sample.

19. The method of claim 1, wherein the sample comprises urine sample, wound exudate, or cultured blood.

20. A method of analyzing a sample for a bacterium, the method comprising:
providing a sample comprising whole cells suspected of including one or more distinct analytes characteristic of a specific bacterium;
providing an analyte-binding material comprising magnetic particles, wherein the magnetic particles have disposed thereon one or more antibodies having antigenic specificities for one or more distinct analytes characteristic of the specific bacterium;
providing a colorimetric sensor comprising a polymerized composition comprising at least one diacetylene-containing polymer and a receptor, wherein the receptor is incorporated in the polymerized composition to form a transducer that provides a color change upon binding with one or more probes and/or analytes;
providing contact between the sample and the analyte-binding material under conditions effective to capture the one or more analytes characteristic of a specific bacterium, if present on the whole cells;
optionally, removing the one or more analytes, if present, from the analyte-binding material; and
subsequent to capture and optional removal of the one or more analytes, subjecting the one or more analytes, if present, to direct or indirect analysis by the colorimetric sensor to analyze for the presence or absence of the specific bacterium.

21. The method of claim 20, wherein the antibodies are selected from the group consisting of MAb-76, MAb-107, affinity-purified RxClf40, affinity-purified GxClf40, MAb 12-9, fragments thereof, and combinations thereof.

22. The method of claim 20 or, wherein the specific bacterium comprises a Gram positive bacterium.

23. The method of claim 22, wherein the specific bacterium comprises Staphylococcus aureus.

24. The method of claim 20, comprising subjecting the one or more analytes, if present, to direct analysis by the colorimetric sensor.

25. The method of claim 20, further comprising the use of one or more probes in an indirect assay.

26. The method of claim 20, further comprising:
providing one or more probes;
providing conditions effective for the probes to bind to the one or more analytes, if present, before capture, after capture, or after optional removal from the solid support material; and
providing contact between the unbound probes and the colorimetric sensor to analyze for the presence or absence of the specific bacterium.

27. The method of claim 26, wherein the one or more probes comprise a polymyxin.

28. The method of claim 20, wherein the magnetic particles comprise at least two portions, wherein one portion of magnetic particles has one antibody specific for one analyte disposed thereon, and a second portion has a different antibody specific for a distinct analyte disposed thereon.

29. The method of claim 20, wherein each magnetic particle of the analyte-binding material has at least two antibodies that bind different analytes disposed thereon.

30. The method of claim 20, wherein the sample comprises urine sample, wound exudate, or cultured blood.

31. A method of analyzing a sample for a Staphylococcus aureus bacterium, the method comprising:
providing a sample comprising whole cells suspected of including one or more distinct analytes characteristic of a Staphylococcus aureus bacterium;
providing an analyte-binding material comprising magnetic particles, wherein the magnetic particles have disposed thereon one or more antibodies having antigenic specificities for one or more distinct analytes characteristic of the Staphylococcus aureus bacterium; wherein the antibodies are selected from the group consisting of MAb-76, MAb-107, affinity-purified RxClf40, affinity-purified GxClf40, MAb 12-9, fragments thereof, and combinations thereof;
providing a colorimetric sensor comprising a polymerized composition comprising at least one diacetylene-containing polymer and a receptor, wherein the receptor is incorporated in the polymerized composition to form a transducer that provides a color change upon binding with one or more probes and/or analytes;
providing contact between the sample and the analyte-binding material under conditions effective to capture the one or more analytes characteristic of a Staphylococcus aureus bacterium, if present on the whole cells;
optionally, removing the one or more analytes, if present, from the analyte-binding material; and
subsequent to capture and optional removal of the one or more analytes, subjecting the one or more analytes, if present, to direct or indirect analysis by the colorimetric sensor to analyze for the presence or absence of the Staphylococcus aureus bacterium.

32. The method of claim 31, wherein the magnetic particles comprise at least two portions, wherein one portion of magnetic particles has one antibody specific for one analyte disposed thereon, and a second portion has a different antibody specific for a distinct analyte disposed thereon.

33. The method of claim 31, wherein each magnetic particle of the analyte-binding material has at least two antibodies that bind different analytes disposed thereon.

34. The method of claim 31, wherein the magnetic particles are blocked to prevent nonspecific binding of a probe in the colorimetric sensor.

35. The method of claim 31, wherein the magnetic particles are blocked with a polymyxin.

36. The method of claim 31, further comprising removing the one or more analytes characteristic of a Staphylococcus aureus bacterium, if present, from the analyte-binding material prior to providing contact with the colorimetric sensor.

37. The method of claim 31, comprising subjecting the one or more analytes, if present, to direct analysis by the colorimetric sensor.

38. The method of claim 31, further comprising the use of one or more probes in an indirect assay.

39. The method of claim 38, wherein the one or more probes comprise polymyxin.

40. The method of claim 31, wherein the sample comprises urine sample, wound exudate, or cultured blood.
